(19) United States
(12) Patent Application Publication
(10) Pub. No.: US 2023/0290480 A1
(43) Pub. Date: Sep. 14, 2023

(54) SYSTEMS AND METHODS FOR CLINICAL TARGET CONTOURING IN RADIOTHERAPY

(71) Applicant: SHANGHAI UNITED IMAGING HEALTHCARE CO., LTD., Shanghai (CN)

(72) Inventors: Supratik BOSE, Houston, TX (US); Lu XU, Shanghai (CN)

(73) Assignee: SHANGHAI UNITED IMAGING HEALTHCARE CO., LTD., Shanghai (CN)

(21) Appl. No.: 18/319,459

(22) Filed: May 17, 2023

Related U.S. Application Data
(63) Continuation of application No. PCT/CN2020/137161, filed on Dec. 17, 2020.

(51) Int. Cl. G16H 20/40; A61N 5/10

(52) U.S. Cl. CPC G16H 20/40; A61N 5/1039

(57) ABSTRACT

A method for clinical target contouring in radiotherapy may include obtaining one or more target images of a subject. The subject may include a target region to which a radiation treatment is directed. The method may also include obtaining a target volume segmentation model having been trained according to a machine learning technique. The method may further include determining boundary information relating to a target volume, the target volume including at least part of the target region based on the one or more target images and the target volume segmentation model.

### CROSS-REFERENCE TO RELATED APPLICATIONS

This application is a continuation of International Application No. PCT/CN2020/137161, filed on Dec. 17, 2020, the contents of which is hereby incorporated by reference.

### TECHNICAL FIELD

The present disclosure generally relates to radiotherapy (RT) technology, and more particularly, to systems and methods for determining boundary information relating to a target volume in radiotherapy.

### BACKGROUND

Radiotherapy technology is widely used in disease treatment. In treatment planning, a target volume (e.g., a gross tumor volume (GTV), a clinical target volume (CTV), or a planning target volume (PTV)) of a subject (e.g., a cancer patient) needs to be segmented and serve as a basis for the generation of a treatment plan of the subject. An accurate segmentation of the target volume is vital in treatment planning.

### SUMMARY

According to an aspect of the present disclosure, a system for clinical target contouring in radiotherapy may be provided. The system may include one or more storage devices and one or more processors configured to communicate with the one or more storage devices. The one or more storage devices may include a set of instructions. When the one or more processors execute the set of instructions, the one or more processors may be directed to cause the system to perform one or more of the following operations. The system may obtain one or more target images of a subject. The subject may include a target region to which a radiation treatment is directed. The system may also obtain a target volume segmentation model having been trained according to a machine learning technique. The system may further determine boundary information relating to a target volume of the subject based on the one or more target images and the target volume segmentation model. The target volume include at least part of the target region.

In some embodiments, the target volume may include at least one of a GTV, a CTV, or a PTV of the subject.

In some embodiments, the target volume segmentation model may have been trained according to a loss function, the loss function being constructed according to a contouring guideline for delineating the target region and one or more organs at risk (OARs) near the target region.

In some embodiments, the target volume may include a CTV of the subject. The target volume segmentation model may include a CTV segmentation model. The loss function may be constructed according to the contouring guideline for delineating the CTV of the target region such that the CTV includes the target region and the OARs near the target region. To determine boundary information relating to a target volume based on the one or more target images and the target volume segmentation model, the system may determine boundary information relating to the CTV of the subject based on the one or more target images and the CTV segmentation model.

In some embodiments, to determine boundary information relating to the CTV of the subject based on the one or more target images and the CTV segmentation model, the system may perform one or more of the following operations. For each of the one or more oars, the system may obtain a segmentation image of the OAR. For each physical point of the subject, the system may obtain position information of the physical point. The system may further determine the boundary information relating to the CTV of the subject by processing the one or more segmentation images, the position information, and the one or more target images of the subject using the CTV segmentation model.

In some embodiments, for each physical point of the subject, the position information of the physical point may include at least one of position information of the physical point along a direction perpendicular to an axial plane of the subject, position information of the physical point along a direction perpendicular to a coronal plane of the subject, or position information of the physical point along a direction perpendicular to a sagittal plane of the subject.

In some embodiments, to generate the CTV segmentation model, the system may obtain a preliminary model and a plurality of training samples. Each of the plurality of training samples may include one or more sample images of a sample subject and ground truth boundary information relating to a sample CTV of the sample subject. The sample CTV may include a sample target region of the sample subject and one or more sample oars near the sample target region. The sample target region may have a same type of lesion as the target region. The system may construct the loss function based on the contouring guideline. The system may generate the CTV segmentation model by training the preliminary model using the plurality of training samples according to the loss function.

In some embodiments, to construct the loss function based on the contouring guideline, the system may convert the contouring guideline into one or more logical constraints. The system may construct the loss function based on the one or more logical constraints.

In some embodiments, to train the preliminary model using the plurality of training samples according to the loss function, the system may perform an iterative operation including one or more iterations. At least one iteration of the one or more iterations may include one or more of the following operations. For each of at least a portion of the plurality of training samples, the system may obtain predicted boundary information of the sample CTV of the training sample based on an updated preliminary model generated in a previous iteration. The system may also determine a value of the loss function based on the predicted boundary information of each of the at least a portion of the plurality of training samples. The system may further determine an assessment result of the updated preliminary model based on the value of the loss function.

In some embodiments, to construct the loss function based on the one or more logical constraints, the system may construct a first loss function for evaluating whether the predicted boundary information of a training sample satisfies the one or more logical constraints and a second loss function for measuring a difference between the predicted boundary information and the ground truth boundary information of a training sample. The system may further construct the loss function based on the first loss function and the second loss function.

In some embodiments, the one or more logical constraints may include a plurality of logical constraints. The first loss function may incorporate a weight of each of the plurality of logical constraints.

In some embodiments, each of the plurality of training samples further include a sample segmentation image of each of the one or more sample oars of the sample subject and sample position information of each sample physical point of the sample subject.

In some embodiments, the one or more processors may be directed to cause the system to perform the following operation. The system may generate a treatment plan directed to the target region based on the boundary information relating to the target volume of the subject.

In some embodiments, to determine boundary information relating to a target volume based on the one or more target images and the target volume segmentation model, the system may determine a model input of the target volume segmentation model based on the one or more target images. The system may also obtain a model output of the target volume segmentation model by inputting the model input into the target volume segmentation model. The system may further determine the boundary information relating to the target volume based on the model output. The model output may include the boundary information relating to the target volume and boundary information relating to one or more oars near the target region.

In some embodiments, to determine boundary information relating to a target volume based on the one or more target images and the target volume segmentation model, the system may obtain boundary information relating to one or more oars near the target region. The system may further determine the boundary information relating to the target volume based on the one or more target images, the target volume segmentation model, and the boundary information relating to the one or more oars.

In some embodiments, to obtain boundary information relating to one or more oars near the target region, the system may obtain one or more OAR segmentation models. The system may further determine the boundary information relating to the one or more oars near the target region based on the one or more target images and the one or more OAR segmentation models.

In some embodiments, to determine the boundary information relating to the target volume based on the one or more target images, the target volume segmentation model, and the boundary information relating to the one or more oars, the system may determine a stage of the target region based on the one or more target images and the boundary information relating to the one or more oars. The system may further determine the boundary information relating to the target volume based on the one or more target images, the target volume segmentation model, the boundary information relating to the one or more oars, and the stage of the target region.

According to another aspect of the present disclosure, a method for clinical target contouring in radiotherapy may be provided. The method may include obtaining one or more target images of a subject. The subject may include a target region to which a radiation treatment is directed. The method may also include obtaining a target volume segmentation model having been trained according to a machine learning technique. The method may further include determining boundary information relating to a target volume, the target volume including at least part of the target region based on the one or more target images and the target volume segmentation model.

According to yet another aspect of the present disclosure, a non-transitory computer readable medium may be provided. The non-transitory computer readable may include at least one set of instructions for clinical target contouring in radiotherapy. When executed by one or more processors of a computing device, the at least one set of instructions may cause the computing device to perform a method. The method may include obtaining one or more target images of a subject. The subject may include a target region to which a radiation treatment is directed. The method may also include obtaining a target volume segmentation model having been trained according to a machine learning technique. The method may further include determining boundary information relating to a target volume, the target volume including at least part of the target region based on the one or more target images and the target volume segmentation model.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary RT system according to some embodiments of the present disclosure;

FIG. 2 is a schematic diagram illustrating an exemplary hardware and/or software components of a computing device according to some embodiments of the present disclosure;

FIG. 3 is a schematic diagram illustrating hardware and/or software components of an exemplary mobile device according to some embodiments of the present disclosure;

FIGS. 4A and 4B are block diagrams illustrating exemplary processing devices according to some embodiments of the present disclosure;

FIG. 5 is a flowchart illustrating an exemplary process for determining boundary information relating to a target volume of a subject according to some embodiments of the present disclosure;

FIG. 6 is a flowchart illustrating an exemplary process for determining boundary information relating to a CTV of a subject according to some embodiments of the present disclosure;

FIG. 7 is a schematic diagram illustrating an exemplary model input of a CTV segmentation model according to some embodiments of the present disclosure;

FIG. 8 is a flowchart illustrating an exemplary process for generating a CTV segmentation model according to some embodiments of the present disclosure;

FIG. 9 is a flowchart illustrating an exemplary process for constructing a loss function according to some embodiments of the present disclosure; and

FIG. 10 is a schematic diagram illustrating an exemplary current iteration of training a preliminary model according to some embodiments of the present disclosure.

FIG. 11 is a schematic diagram illustrating an exemplary process for determining boundary information relating to a target volume of a subject according to some embodiments of the present disclosure;

FIG. 12 is a schematic diagram illustrating an exemplary process for determining boundary information relating to a target volume of a subject according to some embodiments of the present disclosure;

FIG. 13A is schematic diagram illustrating exemplary OAR segmentation images of a subject according to some embodiments of the present disclosure; and

FIG. 13B is a schematic diagram illustrating exemplary CTV segmentation images of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

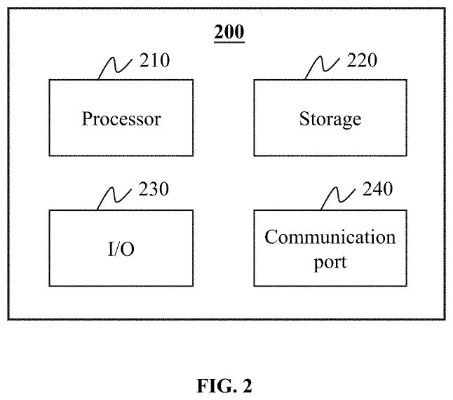

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “anatomical structure” in the present disclosure may refer to gas (e.g., air), liquid (e.g., water), solid (e.g., stone), cell, tissue, organ of a subject, or any combination thereof, which may be displayed in an image (e.g., a second image, or a first image, etc.) and really exist in or on the subject's body. The term “region,” “location,” and “area” in the present disclosure may refer to a location of an anatomical structure shown in the image or an actual location of the anatomical structure existing in or on the subject's body, since the image may indicate the actual location of a certain anatomical structure existing in or on the subject's body. The terms “organ” and “tissue” are used interchangeably referring to a portion of a subject.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood the operations of the flowcharts may be implemented not in order. Conversely, the operations may be implemented in an inverted order, or simultaneously. Moreover, one or more other operations may be added to the flowcharts. One or more operations may be removed from the flowcharts.

Provided herein are systems and components for non-invasive imaging and/or treatment, such as for disease diagnosis, treatment or research purposes. In some embodiments, the systems may include an RT system, a computed tomography (CT) system, an emission computed tomography (ECT) system, an X-ray photography system, magnetic resonance imaging (MRI) system, or the like, or any combination thereof. For illustration purposes, the disclosure describes systems and methods for radiotherapy.

Radiation therapy is widely used in cancer treatment and other treatments. Usually, a treatment plan for a subject (e.g., a cancer patient) is generated before treatment starts based on boundary information of a target volume of the subject. For example, the target volume may include one or more of a GTV, a CTV, and a PTV of the subject. The GTV may include a tumor. The CTV of the subject may include a clinically malignant tissue (e.g., the GTV) and/or a subclinical malignant tissue at a certain probability level. The PTV may include a CTV and an additional margin surrounding the CTV. An accurate determination of the boundary information of the target volume is vital in treatment planning.

However, it is difficult to determine the boundary information of the target volume accurately. For example, the CTV usually includes tissues with a potential tumor or subclinical diseases that are barely detectable in a planning image. In addition, the CTV in the planning image normally has a low contrast visibility and a high noise level, which may lead to ambiguous and blurred boundaries between the CTV and normal tissues in the planning image. Moreover, the delineation of the CTV is highly dependent upon the knowledge and experience of a user (e.g., a physician). Hence, it is significantly more difficult to develop an automated method for contouring a CTV than contouring an organ at risk (OAR). The terms “automatic” and “automated” are used interchangeably referring to methods and systems that analyze information and generates results with little or no direct human intervention.

Recently, some automated methods for CTV contouring based on neural network models have been proposed. For example, a neural network model is trained and used to segment the boundary of a CTV from an image automatically. However, the segmentation results of conventional neural networks have a limited accuracy and clinical acceptability because they don't meet an actual need of a user (e.g., a clinician) and/or contouring guidelines specified by, for example, the Radiation Therapy Oncology Group (RTOG), the Royal Australian and New Zealand College of Radiologists (RANZCR), or other entities. The segmentation results require significant editing by a user before meeting the actual need of the user or the contouring guidelines. Thus, it may be desirable to develop systems and methods for automated CTV contouring by taking contouring guidelines into consideration, thereby improving the efficiency and/or accuracy of CTV contouring.

An aspect of the present disclosure relates to systems and methods for clinical target contouring in radiotherapy. The systems may obtain one or more target images of a subject and a target volume segmentation model. The subject may include a target region to which a radiation treatment is directed. The systems may further determine boundary information relating to the target volume of the subject based on the one or more target images and the target volume segmentation model.

In some embodiments, the target volume may include a CTV of the subject, and the target volume segmentation model may include a CTV segmentation model. The CTV segmentation model may have been trained according to a specifically designed loss function that is constructed according to a contouring guideline for delineating a CTV of the target region such that the CTV includes the target region and one or more OARs near the target region. For example, a contouring guideline for rectal cancer set by the RTOG may be used to construct a loss function of a CTV segmentation model of rectal cancer. In this way, the boundary information of the CTV determined by the CTV segmentation model may better meet the contouring guideline and need less or no editing by users, which in turn, may improve the treatment efficiency and accuracy, and reduce inter-user variations. The systems and methods for CTV contouring may be implemented with reduced or minimal or without user intervention, which is time-saving, more efficient, and accurate.

Another aspect of the present disclosure relates to systems and methods for generating the CTV segmentation model. For example, the systems may obtain a preliminary model and a plurality of training samples. The systems may also construct a loss function based on a contouring guideline for CTV delineation, and generate the CTV segmentation model by training the preliminary model using the plurality of training samples according to the loss function. In some embodiments, in the construction of the loss function, the systems may adopt a particular mechanism to convert the contouring guideline into a one or more logical constraints, and construct the loss function based on the logical constraint(s). The logical constraint(s) may be regarded as a mathematical expression of the contouring guideline, which may include one or more logical operators. Using the particular mechanism, the contouring guideline may be added to any suitable type of machine learning model (e.g., a neural network model).

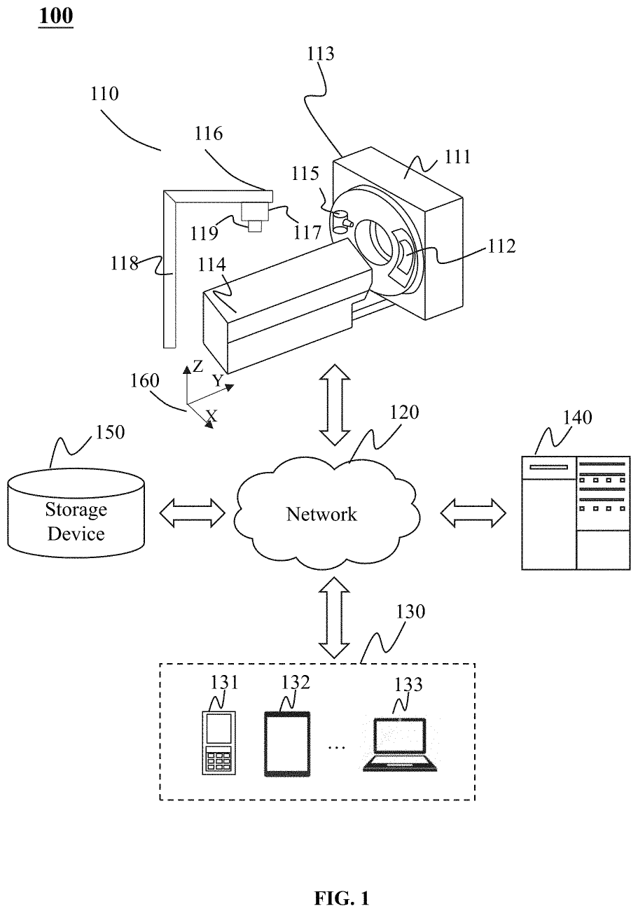

FIG. 1 is a schematic diagram illustrating an exemplary RT system 100 according to some embodiments of the present disclosure. The RT system 100 may include an RT device 110, a network 120, one or more terminals 130, a processing device 140, and a storage device 150. In some embodiments, two or more components of the RT system 100 may be connected to and/or communicate with each other via a wireless connection (e.g., the network 120), a wired connection, or a combination thereof. The connection between the components of the RT system 100 may be variable. Merely by way of example, the RT device 110 may be connected to the processing device 140 through the network 120 or directly. As a further example, the storage device 150 may be connected to the processing device 140 through the network 120 or directly.

The RT device 110 may be configured to deliver a radiotherapy treatment to a subject. For example, the treatment device may deliver one or more radiation beams to a treatment region (e.g., a tumor) of a subject for causing an alleviation of the subject's symptom. A radiation beam may include a plurality of radiation beam lets. In the present disclosure, “object” and “subject” are used interchangeably. The subject may include any biological subject (e.g., a human being, an animal, a plant, or a portion thereof) and/or a non-biological subject (e.g., a phantom). For example, the subject may include a specific portion of a body, such as the head, the thorax, the abdomen, or the like, or a combination thereof, of the subject. In some embodiments, the treatment device may be a conformal radiation therapy device, an image-guided radiation therapy (IGRT) device, an intensity-modulated radiation therapy (IMRT) device, an intensity-modulated arc therapy (IMAT) device, an emission guided radiation therapy (EGRT), or the like.

In some embodiments, the RT device 110 may be an IGRT device configured to acquire image data relating to the subject and perform a radiotherapy treatment on the subject. For example, as illustrated in FIG. 1, the RT device 110 may include an imaging component 113, a treatment component 116, a table (or referred to as couch) 114, or the like. The imaging component 113 may be configured to acquire an image of the subject before radiotherapy treatment, during the radiotherapy treatment, and/or after the radiotherapy treatment. In some embodiments, the imaging component 113 may include a computed tomography (CT) device (e.g., a cone beam CT (CBCT) device, a fan beam CT (FBCT) device), a magnetic resonance imaging (MRI) device, an ultrasound imaging device, a fluoroscopy imaging device, a single-photon emission computed tomography (SPECT) device, a positron emission tomography (PET) device, an X-ray imaging device, or the like, or any combination thereof.

In some embodiments, the imaging component 113 may include an imaging radiation source 115, a detector 112, a gantry 111, or the like. The imaging radiation source 115 and the detector 112 may be mounted on the gantry 111. The imaging radiation source 115 may emit radioactive rays to the subject. The detector 112 may detect radiation events (e.g., x-ray photons, gamma-ray photons) emitted from the imaging region of the imaging component 113. In some embodiments, the detector 112 may include one or more detector units. The detector unit(s) may include a scintillation detector (e.g., a cesium iodide detector, a gadolinium oxysulfide detector), a gas detector, etc. The detector unit(s) may include a single-row detector and/or a multi-rows detector.

The treatment component 116 may be configured to deliver radiation treatment to the subject. The treatment component 116 may include a treatment radiation source 117, a gantry 118, and a collimator 119. The treatment radiation source 117 may be configured to emit treatment radiations towards the subject. In some embodiments, the treatment radiation source 117 may include a linear accelerator (LINAC). The collimator 119 may be configured to control the shape of the treatment radiations generated by the treatment radiation source 117.

In some embodiments, the imaging component 113 may be spaced by a distance from the treatment component 116. In some embodiments, rotation axes of the gantry 111 of the imaging component 113 and the gantry 118 of the treatment component 116 may be the same or different. The subject may be positioned in different positions on the table 114 for imaging and treatment. In some embodiments, the imaging radiation source 115 and the treatment radiation source 117 may be integrated as one radiation source to image and/or treat the subject. In some embodiments, the imaging component 113 and the treatment component 116 may share the same gantry. For example, the treatment radiation source 117 may be mounted on the gantry 111 of the imaging component 113. A subject may be placed on the table 114 for treatment and/or imaging.

The couch 114 may be configured to support the subject to be treated and/or imaged. In some embodiments, the couch 114 may be movable between the treatment component 116 and the imaging component 113 along a Y-axis direction of a coordinate system 160 as shown in FIG. 1. In some embodiments, the couch 114 may be configured to rotate and/or translate along different directions to move the subject to a desired position (e.g., an imaging position under the imaging component 113 for imaging, a treatment position under the treatment component 116 for treatment, etc.).

The network 120 may include any suitable network that can facilitate the exchange of information and/or data for the RT system 100. In some embodiments, one or more components (e.g., the RT device 110, the terminal(s) 130, the processing device 140, the storage device 150, etc.) of the RT system 100 may communicate information and/or data with one or more other components of the RT system 100 via the network 120. For example, the processing device 140 may obtain image data from the RT device 110 via the network 120. As another example, the processing device 140 may obtain user instructions of a user (e.g., a doctor, a radiologist) from the terminal(s) 130 via the network 120. The network 120 may be or include a public network (e.g., the Internet), a private network (e.g., a local area network (LAN)), a wired network, a wireless network (e.g., an 802.11 network, a Wi-Fi network), a frame relay network, a virtual private network (VPN), a satellite network, a telephone network, routers, hubs, switches, server computers, and/or any combination thereof. For example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the RT system 100 may be connected to the network 120 to exchange data and/or information.

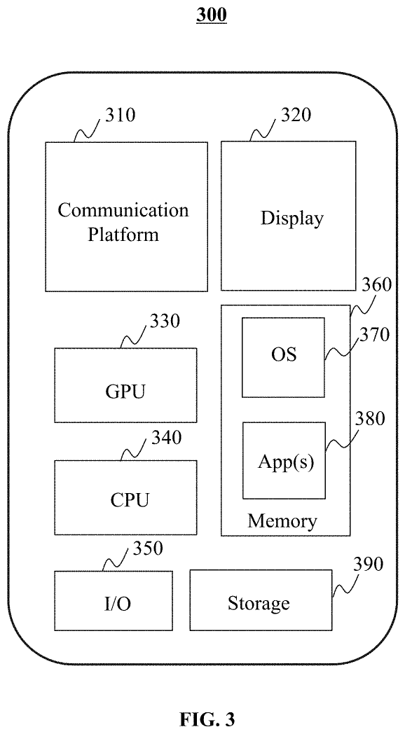

The terminal(s) 130 may enable user interaction between a user and the RT system 100. In some embodiments, the terminal(s) 130 may be connected to and/or communicate with the RT device 110, the processing device 140, and/or the storage device 150. For example, the terminal(s) 130 may display a treatment image of the subject obtained from the processing device 140. In some embodiments, the terminal(s) 130 may include a mobile device 131, a tablet computer 132, a laptop computer 133, or the like, or any combination thereof. In some embodiments, the mobile device 131 may include a smart home device, a wearable device, a mobile device, a virtual reality device, an augmented reality device, or the like, or any combination thereof. Merely by way of example, the terminal(s) 130 may include a mobile device as illustrated in FIG. 3. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, footwear, eyeglasses, a helmet, a watch, clothing, a backpack, a smart accessory, or the like, or any combination thereof. In some embodiments, the mobile device may include a mobile phone, a personal digital assistant (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™, etc. In some embodiments, the terminal(s) 130 may be part of the processing device 140.

The processing device 140 may process information obtained from the RT device 110, the terminal(s) 130, and/or the storage device 150. For example, the processing device 140 may obtain one or more target images (e.g., a CT image, an MRI image) of a subject from one or more components (e.g., the imaging component 113, the terminal(s) 130, the storage device 150) of the RT system 100 or an external source. According to the target image(s) and a target volume segmentation model, the processing device 140 may determine boundary information relating to a target volume of the subject.

In some embodiments, the processing device 140 may generate one or more trained models (e.g., a CTV segmentation model, an OAR segmentation model) that can be used in target volume contouring in radiotherapy. Additionally or alternatively, the processing device 140 may apply the trained model(s) in determining boundary information relating to a target volume. In some embodiments, the trained model(s) may be generated by a processing device, while the application of the model(s) may be performed on a different processing device. In some embodiments, the trained model(s) may be generated by a processing device of a system different from the RT system 100 or a server different from the processing device 140 on which the application of the model(s) is performed. For instance, the trained model(s) may be generated by a first system of a vendor who provides and/or maintains such a model(s), while target volume contouring using the trained model(s) may be performed on a second system of a client of the vendor. In some embodiments, the application of the trained model(s) may be performed online in response to a request for clinical target contouring. In some embodiments, the trained model(s) may be generated offline.

In some embodiments, the trained model(s) may be generated and/or updated (or maintained) by, e.g., the manufacturer of the RT device 110 or a vendor. For instance, the manufacturer or the vendor may load the model(s) into the RT system 100 or a portion thereof (e.g., the processing device 140) before or during the installation of the RT device 110 and/or the processing device 140, and maintain or update the model(s) from time to time (periodically or not). The maintenance or update may be achieved by installing a program stored on a storage device (e.g., a compact disc, a USB drive, etc.) or retrieved from an external source (e.g., a server maintained by the manufacturer or vendor) via the network 150. The program may include a new model (e.g., a new model(s)) or a portion of a model that substitute or supplement a corresponding portion of the model.

In some embodiments, the processing device 140 may be a single server or a server group. The server group may be centralized or distributed. In some embodiments, the processing device 140 may be local or remote. For example, the processing device 140 may access information stored in the RT device 110, the terminal(s) 130, and/or the storage device 150 via the network 120. As another example, the processing device 140 may be directly connected to the RT device 110, the terminal(s) 130 and/or the storage device 150 to access stored information. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented by a computing device 200 having one or more components as illustrated in FIG. 2.

The storage device 150 may store data, instructions, and/or any other information. In some embodiments, the storage device 150 may store data obtained from the RT device 110, the terminal(s) 130, and/or the processing device 140. For example, the storage device 150 may store a target image and/or scan data of the subject. In some embodiments, the storage device 150 may store data and/or instructions that the processing device 140 may execute or use to perform exemplary methods described in the present disclosure. In some embodiments, the storage device 150 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more other components (e.g., the RT device 110, the processing device 140, the terminal(s) 130) of the RT system 100. One or more components of the RT system 100 may access the data and/or instructions stored in the storage device 150 via the network 120. In some embodiments, the storage device 150 may be directly connected to or communicate with one or more other components (e.g., the RT device 110, the processing device 140, the terminal(s) 130) of the RT system 100. In some embodiments, the storage device 150 may be part of the processing device 140.

For illustration purposes, a coordinate system 160 is provided in FIG. 1. The coordinate system 160 may be a Cartesian system including an X-axis, the Y-axis, and a Z-axis. The X-axis and the Y-axis shown in FIG. 1 may be horizontal, and the Z-axis may be vertical. As illustrated, the positive X direction along the X-axis may be from the left side to the right side of the table 114 viewed from the direction facing the front of the RT device 110; the positive Y direction along the Y-axis shown in FIG. 1 may be from the end to the head of the table 114; the positive Z direction along the Z-axis shown in FIG. 1 may be from the lower part to the upper part of the RT device 110.

It should be noted that the above description regarding the RT system 100 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the RT system 100 may include one or more additional components and/or one or more components of the RT system 100 described above may be omitted. For example, the treatment component 116 in the RT device 110 may be omitted. In some embodiments, a component of the RT system 100 may be implemented on two or more sub-components. Two or more components of the RT system 100 may be integrated into a single component. For example, the treatment component 116 in the RT device 110 may be integrated into the imaging component 113.

FIG. 2 is a schematic diagram illustrating exemplary hardware and/or software components of a computing device 200 according to some embodiments of the present disclosure. The computing device 200 may be used to implement any component of the RT system 100 as described herein. For example, the processing device 140 and/or the terminal(s) 130 may be implemented on the computing device 200, respectively, via its hardware, software program, firmware, or a combination thereof. Although only one such computing device is shown, for convenience, the computer functions relating to the RT system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 140 in accordance with techniques described herein. The computer instructions may include, for example, routines, programs, subjects, components, data structures, procedures, modules, and functions, which perform particular functions described herein. For example, the processor 210 may process image data obtained from the RT device 110, the terminal(s) 130, the storage device 150, and/or any other component of the RT system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data obtained from one or more components of the RT system 100. In some embodiments, the storage device 220 may include a mass storage device, a removable storage device, a volatile read-and-write memory, a read-only memory (ROM), or the like, or any combination thereof. In some embodiments, the storage device 220 may store one or more programs and/or instructions to perform exemplary methods described in the present disclosure. For example, the storage device 220 may store a program for the processing device 140 to execute to apply a CTV segmentation model in determining boundary information of a CTV of a subject. As another example, the storage device 220 may store a program for the processing device 140 to execute to generate the CTV segmentation model by model training.

The I/O 230 may input and/or output signals, data, information, etc. In some embodiments, the I/O 230 may enable a user interaction with the processing device 140. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the processing device 140) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), a touch screen), a speaker, a printer, or the like, or a combination thereof.

The communication port 240 may be connected to a network (e.g., the network 120) to facilitate data communications. The communication port 240 may establish connections between the processing device 140 and the RT device 110, the terminal(s) 130, and/or the storage device 150. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of an exemplary mobile device 300 according to some embodiments of the present disclosure. In some embodiments, one or more terminals 130 and/or a processing device 140 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to the RT system 100. User interactions with the information stream may be achieved via the I/O 350 and provided to the processing device 140 and/or other components of the RT system 100 via the network 120.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

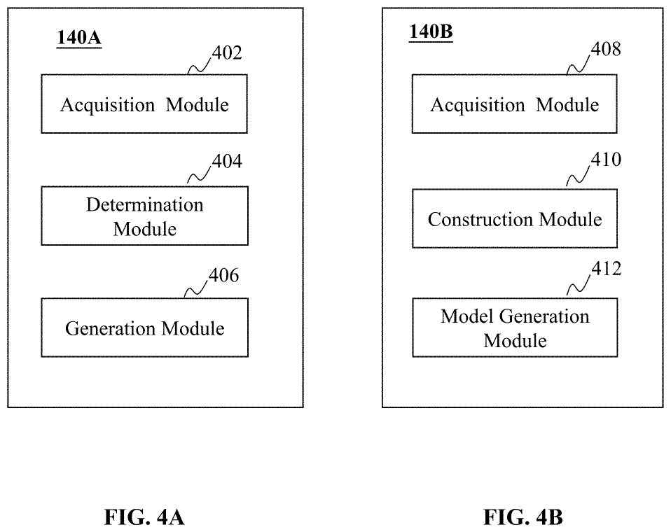

FIGS. 4A and 4B are block diagrams illustrating exemplary processing devices 140A and 140B according to some embodiments of the present disclosure. The processing devices 140A and 140B may be exemplary embodiments of the processing device 140 as described in connection with FIG. 1. In some embodiments, the processing device 140A may be configured to apply one or more machine learning models in determining boundary information relating to a target volume of a subject. The processing device 140B may be configured to generate the one or more machine learning models. In some embodiments, the processing devices 140A and 140B may be respectively implemented on a processing unit (e.g., a processor 210 illustrated in FIG. 2 or a CPU 340 as illustrated in FIG. 3). Merely by way of example, the processing devices 140A may be implemented on a CPU 340 of a terminal device, and the processing device 140B may be implemented on a computing device 200. Alternatively, the processing devices 140A and 140B may be implemented on a same computing device 200 or a same CPU 340. For example, the processing devices 140A and 140B may be implemented on a same computing device 200.

As shown in FIG. 4A, the processing device 140A may include an acquisition module 402, a determination module 404, and a generation module 406.

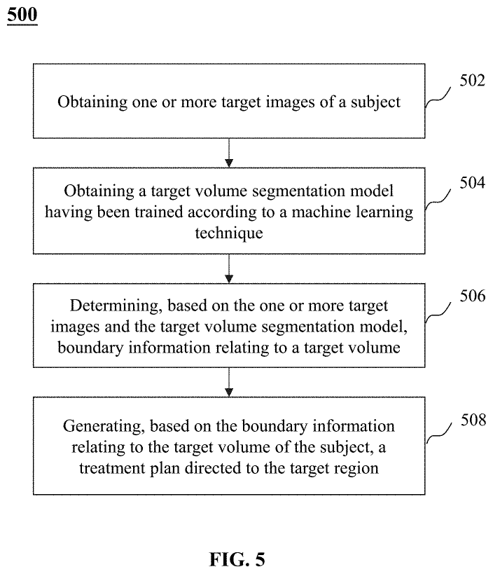

The acquisition module 402 may be configured to obtain information relating to the RT system 100. For example, the acquisition module 402 may obtain one or more target images of a subject. The target image(s) of the subject may include a 2D image (e.g., a slice image), a 3D image, a 4D image (e.g., a series of 3D images over time), and/or any related image data (e.g., scan data, projection data), or the like, or any combination thereof. More descriptions regarding the obtaining of the target image(s) of the subject may be found elsewhere in the present disclosure. See, e.g., operation 502 in FIG. 5 and relevant descriptions thereof. As another example, the acquisition module 402 may obtain a target volume segmentation model having been trained according to a machine learning technique. As used herein, a target volume segmentation model refers to a model (e.g., a machine learning model) or an algorithm for target volume segmentation. More descriptions regarding the obtaining of the target volume segmentation model may be found elsewhere in the present disclosure. See, e.g., operation 504 in FIG. 5 and relevant descriptions thereof.

The determination module 404 may be configured to determine boundary information relating to a target volume of the subject based on the one or more target images and the target volume segmentation model. The target volume may include one or more of the GTV, the CTV, and the PTV of the subject. The boundary information relating to the target volume of the subject may include any information that can be used to identify or localize the boundary of the target volume (e.g., in the physical domain and/or image domain). More descriptions regarding the determination of the boundary information relating to the target volume may be found elsewhere in the present disclosure. See, e.g., operation 506 in FIG. 5 and relevant descriptions thereof.

The generation module 406 may be configured to generate a treatment plan directed to the target region based on the boundary information relating to the target volume of the subject. The treatment plan may describe how the radiotherapy treatment is planned to be performed on the subject to treat the target region. The treatment plan may include information including, e.g., how one or more beams are delivered to the target region of the subject during each treatment session over the course of treatment lasting a certain period of time, e.g., days. More descriptions regarding the generation of the treatment plan may be found elsewhere in the present disclosure. See, e.g., operation 508 in FIG. 5 and relevant descriptions thereof.

As shown in FIG. 4B, the processing device 140B may include an acquisition module 408, a construction module 410, and a model generation module 412.

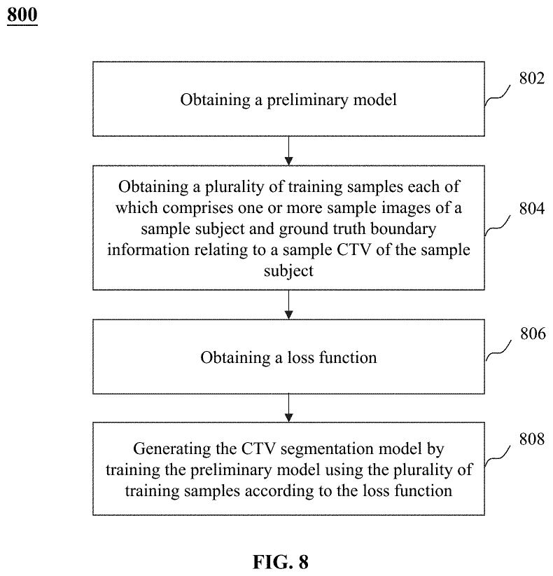

The acquisition module 408 may be configured to obtain one or more training samples and a preliminary model. More descriptions regarding the acquisition of the training samples and the preliminary model may be found elsewhere in the present disclosure. See, e.g., operations 802 and 804 in FIG. 8 and relevant descriptions thereof.

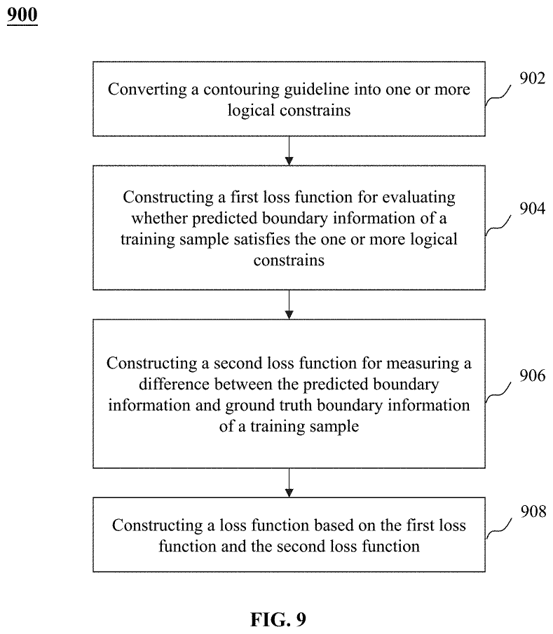

The construction module 410 may be configured to construct a loss function. For example, the construction module 410 may construct a loss function of a CTV segmentation model. In some embodiments, the loss function may be specifically designed based on a contouring guideline for delineating a CTV of the target region such that the CTV includes the target region and one or more OARs near the target region. More descriptions regarding the construction of the loss function may be found elsewhere in the present disclosure. See, e.g., operation 806 in FIG. 8, FIG. 9, and relevant descriptions thereof.

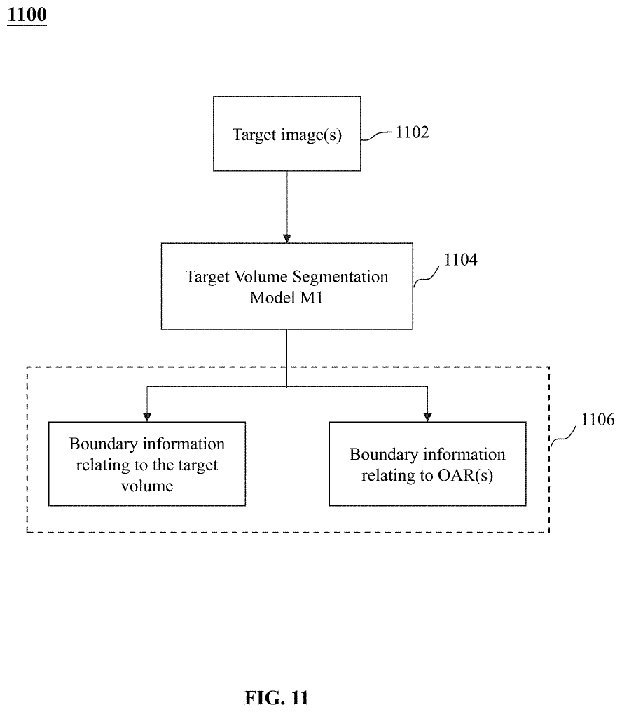

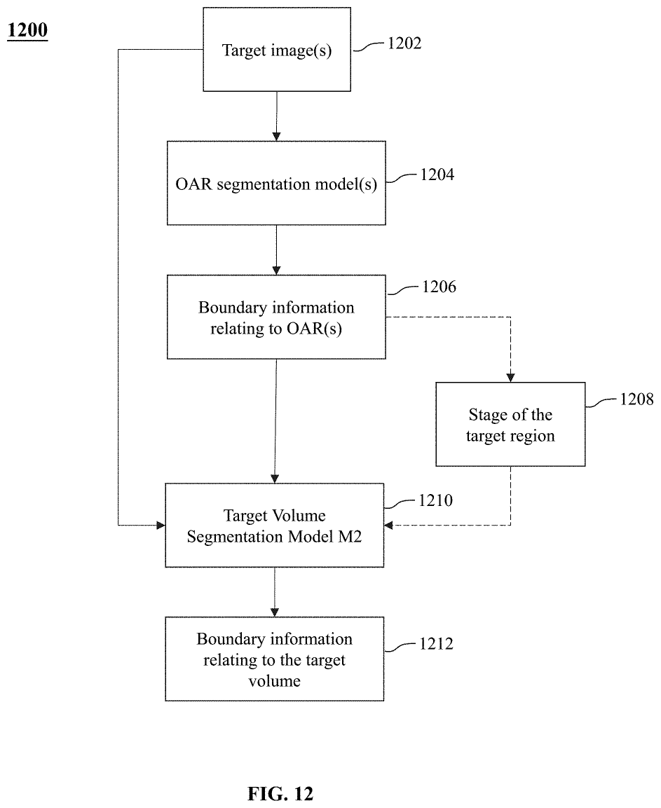

The model generation module 412 may be configured to generate the one or more machine learning models by model training. In some embodiments, the one or more machine learning models may be generated according to a machine learning algorithm. The machine learning algorithm may include but not be limited to an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the one or more machine learning models may be a supervised learning algorithm, a semi-supervised learning algorithm, an unsupervised learning algorithm, or the like. More descriptions regarding the generation of the one or more machine learning models may be found elsewhere in the present disclosure. See, e.g., operation 808 in FIG. 8, operation 1104 in FIG. 11, operation 1210 in FIG. 12, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 140A and/or the processing device 140B may share two or more of the modules, and any one of the modules may be divided into two or more units. For instance, the processing devices 140A and 140B may share a same acquisition module; that is, the acquisition module 402 and the acquisition module 408 are a same module. In some embodiments, the processing device 140A may include one or more additional modules, such as a storage module (not shown) for storing data. In some embodiments, the processing device 140A and the processing device 140B may be integrated into one processing device 140.

FIG. 5 is a flowchart illustrating an exemplary process for determining boundary information relating to a target volume of a subject according to some embodiments of the present disclosure. In some embodiments, process 500 may be executed by the RT system 100. For example, the process 500 may be implemented as a set of instructions (e.g., an application) stored in a storage device (e.g., the storage device 150, the storage device 220, and/or the storage 390). In some embodiments, the processing device 140A (e.g., the processor 210 of the computing device 200, the CPU 340 of the mobile device 300, and/or one or more modules illustrated in FIG. 4A) may execute the set of instructions and may accordingly be directed to perform the process 500.

As used herein, the subject may include a patient, a portion of the patient (e.g., the chest, the breast, and/or the abdomen of the patient), or any organism that needs to be treated by a radiotherapy device (e.g., the RT device 110). The subject may include a target region to which a radiation treatment is directed. For example, the target region may include a region of the subject including at least part of a clinically malignant tissue (e.g., a tumor, a cancer-ridden organ, or a non-cancerous target of radiation therapy). Merely by way of example, the target region may include a tumor, an organ with a tumor or another type of lesion, a tissue with a tumor or another type of lesion, or any combination thereof, that needs to be treated by radiation.

The target volume may include at least part of the target region. For example, the target volume may include at least one of a GTV, a CTV, or a PTV of the subject. Merely by way of example, the target region includes a tumor, and the GTV refers to the tumor itself. A CTV refers to a tissue volume that includes a clinically malignant tissue (i.e., the target region) and/or a subclinical malignant tissue at a certain probability level. The subclinical malignant tissue may include a malignant tissue that has little or no signs or symptoms that are detectable by clinical detection. For example, the subclinical malignant tissue may include one or more OARs near the target region. The OAR(s) may include an organ and/or a tissue that are close to the target region and not intended to be subjected to radiation, but under the risk of radiation damage due to its proximity to the target region. Merely by way of example, if a distance between an organ and the target region is below a threshold distance, the organ may be regarded as an OAR. The threshold distance may include a pixel/voxel distance in the image domain (e.g., 1 pixel/voxel, 2 pixels/voxels, 5 pixels/voxels, etc.) and/or an actual distance in a physical space (e.g., 0.1 cm, 0.2 cm, 0.3 cm, etc.). The threshold distance may be a default setting of the RT system 100, set manually by a user, or adjusted by processing device 140A according to an actual need. A PTV refers to a region surrounding the CTV with an additional margin that allows for variations and/or uncertainties in planning and/or treatment relative to the CTV. For example, the additional margin may allow for variations caused by a positioning error of the subject. The size of the additional margin may be a default setting of the RT system 100, set manually by a user, or adjusted by processing device 140A according to an actual need.

In 502, the processing device 140A (e.g., the acquisition module 402) may obtain one or more target images of the subject.

A target image of the subject may include representations of the target region of the subject and the OAR(s) adjacent to the target region. In some embodiments, the target image(s) of the subject may include a 2D image (e.g., a slice image), a 3D image, a 4D image (e.g., a series of 3D images over time), and/or any related image data (e.g., scan data, projection data), or the like, or any combination thereof. The target image(s) may include one or more grayscale images and/or one or more color images. In some embodiments, the target image(s) may include a medical image generated by a biomedical imaging technique as described elsewhere in this disclosure. For example, the target image(s) may include a CT image (e.g., a cone beam CT (CBCT) image, a fan beam CT (FBCT) image), an MR image, a PET image, an X-ray image, a fluoroscopy image, an ultrasound image, a radiotherapy radiographic image, a SPECT Image, or the like, or a combination thereof. In some embodiments, the target image(s) may include a CT image and an MR image of the subject. In some embodiments, the target image(s) may include a plurality of slice images of the subject. For example, for each of a plurality of axial planes (or referred to as slices) of the subject, a 2D CT image and a 2D MR image of the axial plane may be obtained as the target image(s) of the subject.

In some embodiments, a target image may be generated based on image data acquired using the imaging component 113 of the RT system 100 or an external imaging device. For example, the imaging component 113, such as a CT device, an MRI device, an X-ray device, a PET device, or the like, may be directed to scan the subject or a portion of the subject (e.g., the chest of the subject). The processing device 140A may generate the target image based on image data acquired by the imaging component 113. In some embodiments, a target image may be previously generated and stored in a storage device (e.g., the storage device 150, the storage device 220, the storage 390, or an external source). The processing device 140A may retrieve the target image from the storage device.

In 504, the processing device 140A (e.g., the acquisition module 402) may obtain a target volume segmentation model having been trained according to a machine learning technique.

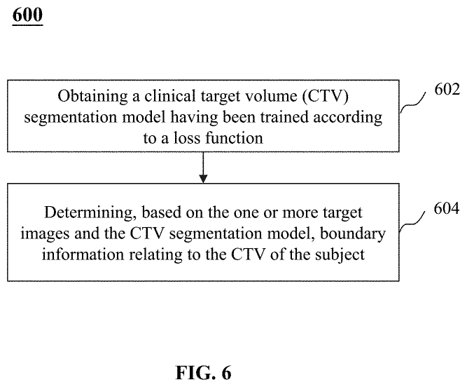

As used herein, a target volume segmentation model refers to a model (e.g., a machine learning model) or an algorithm for target volume segmentation. Merely by way of example, the target volume segmentation model may receive a model input (e.g., the target image(s) and/or other information relating to the subject), and the target volume segmentation model may output boundary information relating to the target volume of the subject. In some embodiments, the target volume segmentation model may output the boundary information relating to the target volume of the subject as well as boundary information relating to the OAR(s) near the target volume. More descriptions regarding the model input and the model output of the target volume segmentation model may be found elsewhere in the present disclosure. See, e.g., FIGS. 6, 11, and 12 and relevant descriptions thereof. In some embodiments, the target volume segmentation model may include a CTV segmentation model. More descriptions regarding the CTV segmentation model may be found elsewhere in the present disclosure. See, e.g., FIGS. 6-10 and relevant descriptions thereof.

In some embodiments, the target volume segmentation model (e.g., the CTV segmentation model) may be a machine learning model. For example, the target volume segmentation model may include a neural network model, such as a Deep Neural Network (DNN) model, a Convolutional Neural Network (CNN) model, a deep dilated residual network (DDRESNET) model, a residual network (ResNet) model, a Recurrent Neural Network (RNN) model, or a Feature Pyramid Network (FPN) model, etc. Exemplary CNN models may include a V-Net model, a U-Net model, or the like, or any combination thereof.

In some embodiments, the processing device 140A (e.g., the acquisition module 402) may obtain the target volume segmentation model from one or more components of the RT system 100 (e.g., the storage device 150, the terminals(s) 130) or an external source via a network (e.g., the network 120). For example, the target volume segmentation model may be previously trained by a computing device (e.g., the processing device 140B), and stored in a storage device (e.g., the storage device 150, the storage device 220, and/or the storage 390) of the RT system 100. The processing device 140A may access the storage device and retrieve the target volume segmentation model. In some embodiments, the target volume segmentation model may be generated according to a machine learning algorithm as described elsewhere in this disclosure (e.g., FIG. 4B and the relevant descriptions).

In 506, the processing device 140A (e.g., the determination module 404) may determine, based on the one or more target images and the target volume segmentation model, boundary information relating to the target volume.

The boundary information relating to the target volume of the subject may include any information that can be used to identify or localize the boundary of the target volume (e.g., in the physical domain and/or image domain). For example, the boundary information may include, for each physical point of the subject or a portion of the subject (e.g., a certain slice of the subject), a classification indicating whether the physical point is inside, or outside, or on the boundary of the target volume of the subject, a probability value that the physical point is inside the boundary of the target volume, a probability value that the physical point is outside the boundary of the target volume, a probability value that the physical point is located on the boundary of the target volume, or the like. As used herein, a physical point of the subject refers to a portion of the subject that corresponds to a voxel or a pixel in a target image or another image of the subject. It should be noted that information relating to a physical point may be also represented by information relating to a pixel or voxel corresponding to the physical point. For example, a probability that a physical point is inside, outside, or on the boundary of the target volume may be represented by a probability that a pixel (or voxel) corresponding to the physical point is inside, outside, or on the boundary of the target volume in a target image, respectively. For illustration purposes, the following descriptions are described with reference to target volume contouring based on the physical points of the subject, and not intended to limit the scope of the present disclosure.

As another example, the boundary information may include a segmentation image of the target volume. In some embodiments, the segmentation image of the target volume may be a binary image in which pixels (or voxels) corresponding to the target volume are displayed in white and other pixels (or voxels) are displayed in black. As another example, the segmentation image of the target volume may be generated by annotating the target volume (e.g., with a specific color) on the target image(s) of the subject. As still another example, the segmentation image of the target volume may be represented as a matrix in which elements having a label of “1” represent physical points of the target volume and elements having a label of “0” represent physical points out of the target volume. As yet another example, the boundary information may include coordinate information of the physical points inside the target volume and/or on the boundary of the target volume.

In some embodiments, the processing device 140A may determine or obtain a model input, and process the model input (e.g., the target image(s) and/or other information relating to the subject) using the target volume segmentation model. Merely by way of example, the model input may be inputted into the target volume segmentation model, and the target volume segmentation model may directly output the boundary information relating to the target volume of the subject. Alternatively, the boundary information relating to the target volume of the subject may be determined based on the output of the target volume segmentation model. For example, the target volume segmentation model may output a probability value that each physical point of the subject is on the boundary of the target volume. The processing device 140A may determine the boundary of the target volume by selecting physical points whose probability values exceed a threshold value.

In some embodiments, the processing device 140A may preprocess the model input and input the processed model input into the target volume segmentation model. For example, the processing device 140A may perform one or more image processing operations, such as an image denoising, an image enhancement, an image smoothing, an image transformation, an image resampling, an image normalization, or the like, or any combination thereof, on one or more images included in the model input. In some embodiments, the target volume segmentation model may have been trained according to a loss function, wherein the loss function may be constructed according to a contouring guideline for delineating the target region and one or more OARs near the target region. In some embodiments, the target volume segmentation model may include the CTV segmentation model, and the processing device 140A may determine boundary information relating to the CTV of the subject by performing process 600 as described in connection with FIG. 6.

In 508, the processing device 140A (e.g., the generation module 406) may generate, based on the boundary information relating to the target volume of the subject, a treatment plan directed to the target region.

The treatment plan may describe how the radiotherapy treatment is planned to be performed on the subject to treat the target region. The treatment plan may include information including, e.g., how one or more beams are delivered to the target region of the subject during each treatment session over the course of treatment lasting a certain period of time, e.g., days. For example, the treatment plan may provide a total dose (e.g., 0.1 Gy, 10 Gy, 50 Gy, 100 Gy, etc.) and a dose distribution in the target region. In some embodiments, the treatment plan may be generated based on the boundary information such that the target volume of the subject may receive an adequate dose (e.g., a dose exceeding a threshold dose) while the dose delivered to the region out of the target volume is minimized during treatment.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 500 may be accomplished with one or more additional operations not described and/or without one or more of the operations discussed above. For example, the process 500 may include an additional transmitting operation to transmit the determined treatment plan to a terminal device (e.g., a terminal 130 of a doctor) for display. As another example, the process 500 may include an additional storing operation to store information and/or data (e.g., the one or more target images, the boundary information relating to the target volume, the treatment plan, etc.) in a storage device (e.g., the storage device 150) disclosed elsewhere in the present disclosure. As yet another example, operation 508 may be omitted.

FIG. 6 is a flowchart illustrating an exemplary process for determining boundary information relating to a CTV of a subject according to some embodiments of the present disclosure. In some embodiments, process 600 may be executed by the RT system 100. For example, the process 600 may be implemented as a set of instructions (e.g., an application) stored in a storage device (e.g., the storage device 150, the storage device 220, and/or the storage 390). In some embodiments, the processing device 140A (e.g., the processor 210 of the computing device 200, the CPU 340 of the mobile device 300, and/or one or more modules illustrated in FIG. 4A) may execute the set of instructions and may accordingly be directed to perform the process 600.

In 602, the processing device 140A (e.g., the acquisition module 402) may obtain a CTV segmentation model having been trained according to a loss function.

A CTV segmentation model refers to a model (e.g., a machine learning model) or an algorithm for CTV segmentation. Merely by way of example, the CTV segmentation model may receive a model input (e.g., the target image(s) and/or other information relating to the subject), and the CTV segmentation model may output boundary information relating to the CTV of the subject.

In some embodiments, the loss function of the CTV segmentation model may be constructed according to a contouring guideline for delineating the CTV of the target region such that the CTV includes the target region and one or more specific OARs near the target region. For example, the contouring guideline may specify that certain margins around the target region of the subject should be included in the CTV of the target region. Merely by way of example, a contouring guideline for rectal cancer may state that in the low pelvic region, the posterior and lateral margin of the CTV of the rectum should extend to the lateral pelvic muscle or bone. In some embodiments, the contouring guideline may be determined by a user (e.g., a doctor or a radiologist) or an organization (e.g., the RTOG, the RANZCR). In some embodiments, different lesion types (e.g., tumor types) may have different contouring guidelines. The loss function of a CTV segmentation model corresponding to a specific lesion type may be constructed according to the contouring guideline of the specific lesion type. More descriptions for the generation of the CTV segmentation model and the construction of the loss function may be found elsewhere in the present disclosure (e.g., FIGS. 8-9 and the descriptions thereof).

In 604, the processing device 140A (e.g., the determination module 404) may determine, based on the one or more target images and the CTV segmentation model, boundary information relating to the CTV of the subject.

The boundary information relating to the CTV of the subject may be similar to that of the target volume as described in connection with operation 508. In some embodiments, the processing device 140A may determine or obtain a model input, and process the model input using the CTV segmentation model. Merely by way of example, the model input may be inputted into the CTV segmentation model, and the CTV segmentation model may directly output the boundary information relating to the CTV of the subject. Alternatively, the boundary information relating to the CTV of the subject may be determined based on the output of the CTV segmentation model. For example, the CTV segmentation model may output a probability value that each physical point of the subject is on the boundary of the CTV. The processing device 140A may determine the boundary of the CTV by selecting physical points whose probability values exceed a threshold value.

In some embodiments, the processing device 140A may preprocess the model input and input the processed model input into the CTV segmentation model. For example, the processing device 140A may perform one or more image processing operations, such as an image denoising, an image enhancement, an image smoothing, an image transformation, an image resampling, an image normalization, or the like, or any combination thereof, on one or more images included in the model input.

In some embodiments, the model input may include the target image(s) of the subject obtained in operation 502. In some embodiments, the target image(s) may include a 3D image of the subject, and the processing device 140A may extract one or more slice images from the 3D image as the model input (or a portion thereof). Additionally or alternatively, the model input may further include a segmentation image of each OAR of the target region, position information of each physical point of the subject, a segmentation image of the target region, a distance between each physical point of the OARs to the boundary of the target region, a segmentation image of a GTV of the subject, a reference segmentation image of the CTV, a stage of the target region, or the like, or any combination thereof.

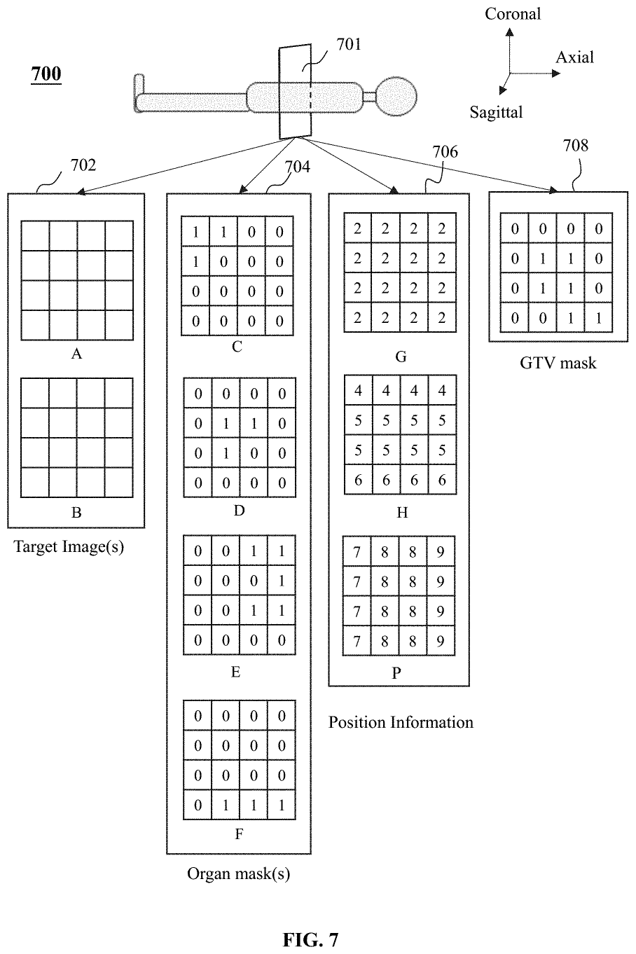

The segmentation image of an OAR may indicate the OAR of the subject segmented from a target image obtained in 502 or another image of the subject. For example, the segmentation image may include a binary segmentation mask of the OAR (e.g., an organ mask 704 as shown in FIG. 7). In the binary segmentation mask, a pixel (or voxel) corresponding to the OAR may be displayed in black, and a pixel (or voxel) corresponding to the remaining region may be displayed in white. As another example, the binary segmentation mask may be represented as a matrix in which elements having a label of “1” represent physical points of the OAR and elements having a label of “0” represent physical points out of the OAR.

For illustration purposes, the generation of the segmentation image of an OAR based on a target image of the subject is described hereinafter. In some embodiments, an OAR may be segmented from the target image manually by a user (e.g., a doctor, an imaging specialist, a technician) by, for example, drawing a bounding box on the target image displayed on a user interface. Alternatively, the segmentation image of an OAR may be generated by the processing device 140A automatically according to an image analysis algorithm (e.g., an image segmentation algorithm). For example, the processing device 140A may perform an image segmentation on the target image using an image segmentation algorithm. Exemplary image segmentation algorithms may include a thresholding segmentation algorithm, a compression-based algorithm, an edge detection algorithm, a machine learning-based segmentation algorithm, or the like, or any combination thereof. Alternatively, the segmentation image of the OAR may be generated by the processing device 140A semi-automatically based on an image analysis algorithm in combination with information provided by a user. Exemplary information provided by the user may include a parameter relating to the image analysis algorithm, a position parameter relating to a region corresponding to the OAR to be segmented, an adjustment to, or rejection or confirmation of a preliminary segmentation result generated by the processing device 140A, etc.

In some embodiments, the processing device 140A may generate the segmentation image of the OAR by processing the target image using one or more machine learning models (e.g., a trained OAR segmentation model as described in connection with FIG. 12). In some embodiments, a plurality of target images may be obtained in 502, and the processing device 140A may generate a segmentation image of the OAR based on each of the target images (or a portion thereof). In some embodiments, the segmentation image of the OAR may be generated by another computing device (e.g., an image processing device of a third party) and transmitted to the processing device 140A. Alternatively, the segmentation image of the OAR may be previously generated and stored in a storage device (e.g., the storage device 150, the storage device 220, the storage 390, or an external source). The processing device 140A may retrieve the segmentation image from the storage device. In some embodiments, the target region may have a plurality of OARs, a portion of which may be mentioned in the contouring guideline. The processing device 140A may generate segmentation images for the OAR(s) that are mentioned in the contouring guideline.

The position information of a physical point of the subject may include, for example, position information of the physical point along a direction perpendicular to an axial plane of the subject (or referred as an axial direction), position information of the physical point along a direction perpendicular to a coronal plane of the subject (or referred to as a coronal direction), or position information of the physical point along a direction perpendicular to a sagittal plane of the subject (or referred to as a sagittal direction), or the like, or any combination thereof.

The position information of the physical point in the axial direction may include, for example, a coordinate of the physical point in the axial direction, a classification regarding where the physical point is located in the axial direction, or the like, or any combination thereof. For example, the subject may be divided into a superior region (e.g., a region near the head of the subject), a middle region (e.g., a region near the abdomen of the subject), and an inferior region (e.g., a region near the feet of the subject) in the axial direction. The position information of the physical point in the axial direction may include a label indicating which region the physical point is located in the axial direction, for example, a label “1” corresponding to the inferior region, a label “2” corresponding to the middle region, and a label “3” corresponding to the superior region.

The position information of the physical point in the coronal direction may include, for example, a coordinate of the physical point in the coronal direction, a classification regarding where the physical point is located in the coronal direction, or the like, or any combination thereof. For example, the subject may be divided into a front region (e.g., a region including the breast of the subject), a first central region (e.g., a region in the middle of the subject), and a back region (e.g., a region including the back of the subject) in the coronal direction. The position information of the physical point in the coronal direction may include a label indicating which region the physical point is located in the coronal direction, for example, a label “4” corresponding to the front region, a label “5” corresponding to the first central region, and a label “6” corresponding to the back region.

The position information of the physical point in the sagittal direction may include, for example, a coordinate of the physical point in the sagittal direction, a classification regarding where the physical point is located in the sagittal direction, or the like, or any combination thereof. For example, the subject may be divided into a left region (e.g., a region including the left arm of the subject), a second central region (e.g., a region including the chest of the subject), and a right region (e.g., a region including the right arm of the subject) in the sagittal direction. The position information of the physical point in the sagittal direction may include a label indicating which region the physical point is located in the sagittal direction, for example, a label “7” corresponding to the right region, a label “8” corresponding to the second central region, and a label “9” corresponding to the left region.

In some embodiments, the division of the subject in the axial direction, the coronal direction, and/or the sagittal direction may be performed according to a default setting of the RT system 100, or manually by a user, or by the processing device 140A according to an actual need. Merely by way of example, the subject may be evenly divided into three regions along the axial direction, wherein the three regions may be designated as the superior, the middle, and the inferior region, respectively. As another example, the subject may be evenly divided into four regions along the sagittal direction, among which the region at the right side of the subject is designated as the right region, the region at the left side of the subject is designated as the left region, and the two regions in the middle of the subject are designated as the second central region.

The segmentation image of the target region may indicate the target region of the subject segmented from a target image obtained in 502 or another image of the subject. The generation of the segmentation image of the target region may be performed in a similar manner as that of the segmentation image of an OAR, and the descriptions thereof are not repeated here. The distance between a physical point of an OAR to the boundary of the target region may include an actual distance in the physical space or a distance in the image domain expressed in terms of, e.g., pixel/voxel number (count), length, etc.

The segmentation image of the GTV (or referred to as a GTV mask) may indicate the GTV of the subject segmented from a target image obtained in 502 or another image of the subject. The generation of the segmentation image of the GTV may be performed in a similar manner as that of the segmentation image of an OAR, and the descriptions thereof are not repeated here.

The reference segmentation image of the CTV may be generated based on a segmentation image of the GTV (e.g., the tumor). Merely by way of example, the reference segmentation image may be generated by dilating the boundary of the GTV in the segmentation image of the GTV in different directions (e.g., the axial direction, the coronal direction, and the sagittal direction). In some embodiments, the dilation of the boundary of the GTV may be performed according to the contouring guideline.

The stage of the target region may reflect a severity of a lesion in the target region. For example, the stage of a tumor may reflect a malignant level of the tumor. In some embodiments, the processing device 140A may determine the stage of the target region based on, for example, the size, the position, whether the tumor has spread to nearby organs, or the like, or any combination thereof. Merely by way of example, the processing device 140A may determine the stage of a tumor (e.g., stage 0, stage 1, stage 2, stage 3, stage 4, etc.) according to a TNM staging system. As another example, the processing device 140A may determine the stage of a tumor based on the target image(s) of the tumor, one or more segmentation images of one or more OARs near the tumor, and/or other information (e.g., diagnostic information) regarding the tumor using a stage determination model. The stage determination model may be a model (e.g., a machine learning model) or an algorithm configured for stage determination based on its input.

In some embodiments, in operation 602, the processing device 140A may determine the stage of the target region, and obtain or generate a specific CTV segmentation model corresponding to the stage of the target region. For example, tumors having different stages may have different contouring guidelines, and a plurality of CTV segmentation models corresponding to different stages may be generated and stored in a storage device (e.g., the storage device 150). The processing device 140A may select the specific CTV segmentation model corresponding to the stage of the target region from the plurality of CTV segmentation models, and utilize the specific CTV segmentation model for determining the boundary information of the CTV of the subject. As another example, the processing device 140A may obtain a contouring guideline corresponding to the stage of the target region, and further generate the specific CTV segmentation model according to the obtained contouring guideline.

FIG. 7 is a schematic diagram illustrating an exemplary model input 700 of a CTV segmentation model according to some embodiments of the present disclosure. As shown in FIG. 7, the model input of the CTV segmentation model may include one or more target images 702 of a slice 701 of a patient, one or more organ masks 704 of one or more OARs of the patient, position information 706 relating to physical points of the slice 701, and a GTV mask 708 of the patient.

Merely by way of example, the target image(s) 702 may include a CT slice image A and an MR slice image B of the slice 701. The organ mask(s) 704 (or referred to as segmentation image(s)) may include organ masks C, D, E, and F of four OARs of the patient. In an organ mask of an OAR, a region including elements labeled by “1” corresponds to the OAR of the patient, and the remaining region (i.e., the region including elements labeled by “0”) is a background region. In some embodiments, an organ mask may reflect boundary information of multiple OARs. For example, elements corresponding to different OARs may be annotated with different labels (e.g., “1,” “2,” and “3”) in the organ mask. Similar to an organ mask, in the GTV mask 708, a region including elements labeled by “1” corresponds to the GTV of the patient, and the remaining region (i.e., the region including elements labeled by “0”) is a background region.

The position information 706 of the physical points in the slice 701 may include a position map G corresponding to the axial direction as shown in FIG. 7, a position map H corresponding to the coronal direction as shown in FIG. 7, and a position map P corresponding to the sagittal direction as shown in FIG. 7. In the position map G, a label “1” of a physical point indicates that the physical point is located in an inferior region of the patient, a label “2” indicates that the physical point is located in a middle region of the patient, and a label “3” indicates that the physical point is located in a superior region of the patient. In the position map H, a label “4” of a physical point indicates that the physical point is located in a front region of the patient, a label “5” indicates that the physical point is located in a first central region of the patient, and a label “6” indicates that the physical point is located in a back region of the patient. In the position map P, a label “7” of a physical point indicates that the physical point is located in a right region of the patient, a label “8” indicates that the physical point is located in a second central region of the patient, and a label “9” indicates that the physical point is located in the left region of the patient.

As shown in FIG. 7, all physical points in the slice 701 are located in the middle region of the patient in the axial direction, a quarter of the physical points are located in the front region in the coronal direction, half of the physical points are located in the first central region in the coronal direction, a quarter of the physical points are located in the back region in the coronal direction, a quarter of the physical points are located in left region in the sagittal direction, half of the physical points are located in the second central region in the sagittal direction, and a quarter of the physical points are located in the right region in the sagittal direction.

In some embodiments, if a target image of the patient is a 2D grayscale image, the target image may be represented as a matrix of dimensions w×h, wherein w refers to the width of the target image and h refers to the height of the target image. If the target image is a 2D color image, the target image may be represented as a matrix of dimensions w×h×C, wherein C refers to the count of RGB channels. Normally, a color image normally has three RGB channels corresponding to the red, the green, and the blue, respectively. When the 2D color image is inputted into the CTV segmentation model, a 2D convolution layer may receive the 2D color image as input and output an activation map of dimensions w′×h′×C′, wherein w′ refers to the width of the activation map, h′ refers to the height of the activation map, and C′ refers to the count of output channels.

In some embodiments, for each of one or more slices of the patient, one or more slice images of the slice may be obtained or generated (e.g., extracted from a 3D target image of the patient). The total count of the slice image(s) of the slice(s) is denoted as t, the width of each slice image is denoted as w, the height of each slice image is denoted as h, and the count of RGB channels of each slice image is denoted as C. In such cases, the input dimension of the CTV segmentation model may be (w×h×t)×C.

In some embodiments, the input of the CTV segmentation model may include a plurality of channels (or referred to as feature dimensions) with respect to each slice of the patient. For example, it is assumed that the contouring guideline of the target region of the patient specifies that a set of OARs (O₁, O₂, to O_{N}) needs to be considered in the determination of the CTV of the target region. The input of the CTV segmentation model may include (M+N+4) channels for each slice of the patient as shown in Table 1 below, wherein M refers to the count of imaging modalities used to acquire the slice image(s), and N refers to the count of the OARs, and 3 refers to the count of channels relating to position information of the slice.

TABLE 1
Exemplary channels of a slice of a patient

| Channel index | Information encoded in the channel |
|---|---|
| Channel 0 | Slice image of the slice acquired by a first |
| | imaging modality (e.g., CT) |
| Channel 1 | Slice image of the slice acquired by a second |
| | imaging modality (e.g., MRI) |
| . . . | . . . |
| Channel (M − 1) | Slice image of the slice acquired by a (M − 1)^{th} |
| | imaging modality |
| Channel M to | Segmentation images of the OARs O₁ to O_{N} in the |
| Channel M + N − 1 | slice |
| Channel M + N | Segmentation image of the target region in the slice |
| Channel M + N + 1 | Position map including position information of each |
| | physical point of the slice in the axial direction |
| Channel M + N + 2 | Position map including position information of each |
| | physical point of the slice in the coronal direction |
| Channel M + N + 3 | Position map including position information of each |
| | physical point of the slice in the sagittal direction |

It should be noted that the above description regarding the model input of the CTV segmentation model is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. Merely by way of example, the input of the CTV segmentation model may include other information relating to the patient. Additionally or alternatively, some information discussed above may be omitted. As another example, a target image, an organ mask, and/or a position map of the slice 701 of the patient may be represented in any other form.

FIG. 8 is a flowchart illustrating an exemplary process for generating a CTV segmentation model according to some embodiments of the present disclosure. In some embodiments, process 800 may be executed by the RT system 100. For example, the process 800 may be implemented as a set of instructions (e.g., an application) stored in a storage device (e.g., the storage device 150, the storage device 220, and/or the storage 390). In some embodiments, the processing device 140B (e.g., the processor 210 of the computing device 200, the CPU 340 of the mobile device 300, and/or one or more modules illustrated in FIG. 4B) may execute the set of instructions and may accordingly be directed to perform the process 800. In some embodiments, the CTV segmentation model described in connection with operation 602 in FIG. 6 may be obtained according to the process 800. In some embodiments, the process 800 may be performed by another device or system other than the RT system 100, e.g., a device or system of a vendor or a manufacturer of the CTV segmentation model. For illustration purposes, the implementation of the process 800 by the processing device 140B is described as an example.

In 802, the processing device 140B (e.g., the acquisition module 408) may obtain a preliminary model.

The preliminary model refers to a model to be trained. The preliminary model may be of any type of model (e.g., a machine learning model) as described elsewhere in this disclosure (e.g., FIG. 5 and the relevant descriptions). In some embodiments, the processing device 140B may obtain the preliminary model from one or more components of the RT system 100 (e.g., the storage device 150, the terminals(s) 130) or an external source (e.g., a database of a third-party) via a network (e.g., the network 120).

The preliminary model may include a plurality of model parameters. For example, the preliminary model may be a CNN model and exemplary model parameters of the preliminary model may include the number (or count) of layers, the number (or count) of kernels, a kernel size, a stride, a padding of each convolutional layer, or the like, or any combination thereof. Before training, the model parameters of the third preliminary model may have their respective initial values. For example, the processing device 140B may initialize parameter values of the model parameters of the third preliminary model.

In 804, the processing device 140B (e.g., the acquisition module 408) may obtain a plurality of training samples.

Each of the training samples may include one or more sample images of a sample subject and ground truth boundary information relating to a sample CTV of the sample subject, wherein the sample CTV may include a sample target region of the sample subject and one or more sample OARs near the sample target region. The sample target region of a sample subject may be a region having a certain type of lesion that needs to be treated by a radiation treatment.

In some embodiments, the sample target regions of different training samples may have a same type of lesion. Two lesions may be deemed as belonging to a same type if they, for example, are located at a same organ or tissue, have same or similar symptoms, or the like, or any combination thereof. For example, the sample target regions of different training samples may both have liver cancer. Since different lesion types may have different contouring guidelines, different CTV segmentation models may need to be generated for different lesion types. For example, to generate a CTV segmentation model used in CTV contouring for stomach cancer, a plurality of training samples of a plurality of sample patients with stomach cancer may be obtained, and the sample target region of each sample patient may include the stomach of the sample patient. In some embodiments, the sample target region of each training sample may have a same type of lesion (e.g., tumor) as the target region of the subject as described in connection with FIG. 5. A sample OAR of the sample subject may be similar to an OAR of the target region as described in connection with FIG. 5.

The sample image(s) of a sample subject may include, for example, a CT image, an MR image, a PET image, or the like, or any combination thereof, of the sample subject. The sample image(s) of the sample subject may be similar to the target image(s) of the subject as described in connection with operation 502. The ground truth boundary information relating to the sample CTV of the sample subject refers to boundary information that can be used to identify or localize the boundary of the sample CTV. The boundary of the sample CTV may be determined or confirmed by a user according to a contouring guideline for delineating a CTV of the sample target region of the sample subject. For example, a sample image of a sample patient with lung cancer may be displayed on a user terminal, and a doctor may draw the boundary of the CTV of the lungs of the sample patient on the sample image according to a contouring guideline corresponding to lung cancer. As another example, a preliminary boundary of the CTV of the lungs of the sample patient may be determined by a computing device, and the doctor may adjust the preliminary boundary so that it meets the contouring guideline corresponding to lung cancer.

In some embodiments, a training sample of a sample subject may further include a sample segmentation image of each sample OAR of the sample subject, sample position information of each sample physical point of the sample subject, a sample segmentation image of the sample target region of the sample subject, a sample distance between each sample physical point of a sample OAR and the boundary of the sample target region of the sample subject, or the like, or any combination thereof. The sample segmentation image of a sample OAR may be similar to a segmentation image of an OAR as described in connection with operation 604. The sample position information of a sample physical point of the sample subject may be similar to position information of a physical point of the subject as described in connection with operation 604. For example, the sample position information of a sample physical point of the sample subject may include sample position information along the axial direction of the sample subject, sample position information along the coronal direction of the sample subject, sample position information along the sagittal direction of the sample subject, or the like, or any combination thereof.

In some embodiments, the processing device 140B may obtain a training sample (or a portion thereof) from one or more components of the RT system 100 (e.g., the storage device 150, the terminals(s) 130) or an external source (e.g., a database of a third-party) via a network (e.g., the network 120). Alternatively, the training sample (or a portion thereof) may be generated by the processing device 140B. Merely by way of example, the processing device 140B may generate a sample segmentation image of a sample OAR of a sample subject based on a sample image of the sample subject.

In some embodiments, the processing device 140B may determine a sample model input to be inputted into the preliminary model (or an updated preliminary model) based on the training samples. For example, for each sample physical point of a sample subject of a training sample, the processing device 140B may construct a first 5D tensor as a sample model input with respect to the sample physical point. The first 5D tensor of a sample physical point may include 5 dimensions including a batch index (e.g., an identification of the training sample to which the sample physical point belongs), a channel index, a slice index, a row index, and a column index. The batch index, the slice index, the row index, and the column index may be used to identify the physical location of the sample physical point. The sample physical point may have a plurality of first channels (or referred to as feature dimensions) to be processed by the (updated) preliminary model, and the channel index may be used to identify a specific channel among the first channels.

In some embodiments, the first channels of the sample physical point may be similar to the channels of a slice of a patient as shown in Table. 1. Merely by way of example, the sample physical point may be located in a specific slice of a sample subject, and the first channels of the sample physical point may include a pixel/voxel value of the sample physical point in a slice image of the specific slice, a label of the sample physical point in a sample segmentation image of a sample OAR of the sample subject, sample position information of the sample physical point in the coronal direction of the sample subject, sample position information of the sample physical point in the axial direction of the sample subject, sample position information of the sample physical point in the sagittal direction of the sample subject, or the like, or any combination thereof. It should be noted that the sample model input with respect to the sample physical point as aforementioned is merely provided for illustration purposes, and not intended to limit the scope of the present disclosure. The sample model input with respect to the sample physical point may be represented in any form, e.g., a vector with any count of dimensions, a flat array, or the like.

In 806, the processing device 140B (e.g., the construction module 410) may obtain a loss function.

The loss function may evaluate the accuracy of the preliminary model or an updated preliminary model updated from the preliminary model. For example, the loss function may be used to evaluate whether a predicted result (e.g., predicted boundary information) outputted by the updated preliminary model satisfies the contouring guideline and/or whether the predicted result is consistent with the ground truth boundary information of the training samples. In some embodiments, the loss function may be specifically designed based on the contouring guideline such that the (updated) preliminary model may learn a CTV contouring mechanism that meets the contouring guideline. In some embodiments, the processing device 140B may convert the contouring guideline into one or more logical constraints, and construct the loss function based on the one or more logical constraints. More descriptions regarding the construction of the loss function may be found elsewhere in the present disclosure. See, e.g., FIG. 9 and relevant descriptions thereof.

In 808, the processing device 140B (e.g., the model generation module 412) may generate the CTV segmentation model by training the preliminary model using the plurality of training samples according to the loss function.

During the training process of the preliminary model, the processing device 140B may update the parameter values of the model parameters of the preliminary model from the initial values to trained values according to the loss function, so as to generate the CTV segmentation model. For example, the training of the preliminary model may include an iterative operation including one or more iterations. For illustration purposes, the implementation of a current iteration is described hereinafter. The current iteration may be performed based on at least a portion of the training samples. In some embodiments, a same set or different sets of training samples may be used in different iterations in training the preliminary model. For the convenience of descriptions, the at least a portion of the training samples used in the current iteration is referred to as target training sample(s).

Merely by way of example, in the current iteration, for each target training sample, the processing device 140B may obtain predicted boundary information of the sample CTV of the target training sample based on an updated preliminary model generated in a previous iteration. For example, for a target training sample, the processing device 140B may generate a sample model input and input the sample model input into the updated preliminary model. The updated preliminary model may output the predicted boundary information of the target training sample, or other information that needs to be processed by the processing device 140B to generate the predicted boundary information. If the current iteration is the first iteration during the training process, the processing device 140B may obtain the predicted boundary information of the target training sample based on the original preliminary model.

In some embodiments, as described in connection with operation 804, the processing device 140B may determine a sample model input with respect to each sample physical point of a training sample. The sample model input with respect to a sample physical point may be represented as a first 5D tensor including dimensions of a batch index, a channel index, a slice index, a row index, and a column index. The updated preliminary model may be configured to generate a sample output with respect to the sample physical point. For example, the sample output may be represented as a second 5D tensor including the same dimensions as the first 5D tensor. The channel index of the second 5D tensor may be used to identify a specific second channel (or referred to as a feature dimension) of the sample physical point outputted by the updated preliminary model.

The processing device 140B may further determine the value of the loss function based on the predicted boundary information of each target training sample. The processing device 140B may then determine an assessment result of the updated preliminary model based on the value of the loss function. The assessment result may indicate whether the updated preliminary model is sufficiently trained. For example, the processing device 140B may determine whether a termination condition is satisfied in the current iteration based on the value of the loss function. An exemplary termination condition may be that the value of the loss function in the current iteration is less than a threshold value, a difference between the values of the loss function obtained in a previous iteration and the current iteration (or among the values of the loss function within a certain number or count of successive iterations) is less than a certain threshold, or the like, or any combination thereof. Other exemplary termination conditions may include that a maximum number (or count) of iterations has been performed.

In response to determining that the termination condition is not satisfied in the current iteration, the processing device 140B may determine that the updated preliminary model is not sufficiently trained, and further update the updated preliminary model based on the value of the loss function. Merely by way of example, the processing device 140B may update at least some of the parameter values of the updated preliminary model according to a backpropagation algorithm, e.g., a stochastic gradient descent backpropagation algorithm. The processing device 140B may further perform a next iteration until the termination condition is satisfied. In response to determining that the termination condition is satisfied in the current iteration, the processing device 140B may determine that the updated preliminary model is sufficiently trained and terminate the training process. The updated preliminary model may be designated as the CTV segmentation model.

FIG. 9 is a flowchart illustrating an exemplary process for constructing a loss function of a CTV segmentation model according to some embodiments of the present disclosure. In some embodiments, one or more operations of the process 900 may be performed to provide the loss function involved in operation 806 as described in connection with FIG. 8.

In 902, the processing device 140B (e.g., the construction module 410) may convert the contouring guideline into one or more logical constraints.

A logical constraint may be regarded as a mathematical expression of the contouring guideline. For example, the logical constraint may include one or more logical operators, such as, a logical-and, a logical-or, negation (i.e., not), a conditional statement (e.g., “if . . . then . . . ”) to express the contouring guideline. In some embodiments, the logical constraint(s) may be used to evaluate the accuracy of the preliminary model or an updated preliminary model updated from the preliminary model during model training. For example, the logical constraint(s) may evaluate whether a sample physical point predicted to be a boundary physical point of a sample CTV meets the contouring guideline.

For illustration purposes, exemplary embodiments of converting a contouring guideline into logical constraint(s) are provided hereinafter. Merely by way of example, the one or more logical constraints may include a plurality of logical operators as shown in Equations (1) to (5) below:

A&B=max{A+B−1,0}, (1)

A OR B=min{A+B,1}. (2)

A1\hat{ }A2\hat{ }An=ΣAi/N, (3)=

[symbol]

=1−A, (4)

A=>B= [symbol] A OR B, (5)

where A and B refer to two data fields to be analyzed, “&” and “\hat{ }” may have a similar function to a logical-and operator, “&” may be used as a selection operator, and “\hat{ }” may be an averaging operator.

For example, a first contouring guideline for rectal cancer may state that in the low pelvic region, the posterior and lateral margin of the CTV of the rectum should extend to the lateral pelvic muscle or bone. The processing device 140B may convert the first contouring guideline into a first logical constraint as shown in Equation (6) as below:

[Inferior==axial(X[b,M+N+1,d,r,c])]& [Boundary_CTV==class(Y[b,0,d,r,c])]& [Back==coronal(X[b,M+N+2,d,r,c]) OR Left==sagittal(X[b,M+N+3,d,r,c]) OR Right==sagittal(X[b,M+N+3,d,r,c])]=>[1==X[b,p1,d,r,c] OR 1==X[b,p2,d,r,c]], (6)

where b denotes a batch index of a sample physical point of a sample subject, (M+N+1) denotes a channel index corresponding to position information in the axial direction, (M+N+2) denotes a channel index corresponding to position information in the coronal direction, (M+N+3) denotes a channel index corresponding to position information in the sagittal direction, d denotes a slice index of a sample slice where the sample physical point locates, r denotes a row index of the sample physical point in the sample slice, c denotes a column index of the sample physical point in the sample slice, p1 denotes a channel index for the pelvic bone, and p2 denotes a channel index for the pelvic muscle.

In the first logical constraint, the first clause [Inferior==axial(X[b, M+N+1, d, r, c])] may be used to determine whether the sample physical point is located in the inferior region of the sample subject in the axial direction, which may be equal to 1 if the sample physical point is located in the inferior region and 0 if the sample physical point is not located in the inferior region. The second clause [Boundary_CTV==class (Y [b, 0, d, r, c])] may be used to determine whether the sample physical point is predicted to be on the boundary of the sample CTV of the rectum, which may be equal to 1 if the sample physical point is predicted to be on the boundary and 0 if the sample physical point is predicted to be not on the boundary. The third clause [Back==coronal(X[b, M+N+2, d, r, c]) OR Left==sagittal(X[b, M+N+3, d, r, c]) OR Right==sagittal(X[b, M+N+3, d, r, c])] may be used to determine whether the sample physical point is located in the back region of the sample subject in the coronal direction, or the left region or the right region of the sample subject in the sagittal direction, which may be equal to 1 if the sample physical point is located in the back region, or the left region, or the right region, and equal to 0 if the sample physical point is not located in the back region, the left region, or the right region. The fourth clause [1==X[b, p1, d, r, c] OR 1==X[b, p2, d, r, c]] may be used to determine whether the sample physical point belongs to the pelvic bone or the pelvic muscle of the sample subject, which may be equal to 1 if the sample physical point belongs to the pelvic bone or the pelvic muscle, and equal to 0 if the sample physical point belongs to neither the pelvic bone nor the pelvic muscle.

The logical operator “=>” in the first logical constraint may be used to make an assertion that if the sample physical point is in the inferior region of the sample subject in the axial direction, and is also located either in the back region in the coronal direction or in the left or right region in the sagittal direction, and if the sample physical point is predicted to be a boundary physical point of the sample CTV of the rectum, the sample physical point also belongs to the pelvic bone or the pelvic muscle. The value of the second clause [Boundary_CTV==class(Y[b, 0, d, r, c]] may be determined based on a predicted result regarding the sample physical point outputted by the preliminary model or the updated preliminary model during model training. If the first logical constraint is equal to 1, the assertion may be deemed to be true and the predicted result regarding the sample physical point satisfies the first contouring guideline for rectal cancer; if the first logical constraint is equal to 0, the assertion may be deemed to be false and the predicted result regarding the sample physical point doesn't satisfy the first contouring guideline. Merely by way of example, only if all values of the first clause, the second clause, and the third clause are equal to 1, the left side of the logical operator “=>” may be equal to 1, otherwise, the left side may be equal to 0. If the sample physical point belongs to neither the pelvic muscle nor the pelvic bone, the right side of the logical operator “=>” may be equal to 0, otherwise, the right side may be equal to 1. If the left side is equal to 1 and the right is equal to 0, the first logical constraint may be equal to 0 and the assertion is deemed to be false. In other words, a sample physical point that doesn't belong to the pelvic muscle or the pelvic bone is determined to be on the boundary of the sample CTV of the rectum by the (updated) preliminary model, indicating that the (updated) preliminary model outputs a false prediction and needs to be “penalized.” If the left side is equal to 1 and the right is equal to 1, the first logical constraint may be equal to 1 and the assertion is deemed to be true. In other words, a sample physical point that belongs to the pelvic muscle or the pelvic bone is determined to be on the boundary of the CTV of the rectum by the (updated) preliminary model, indicating that the (updated) preliminary model outputs a true prediction and does not need to be “penalized.”

As another example, a second contouring guideline for rectal cancer may state that in the middle pelvic region, the CTV of the rectum should cover rectum, mesorectum, internal iliac vessels, and presacral space. The processing device 140B may convert the second contouring guideline into a second logical constraint as shown in Equation (7) as below:

[Middle==axial(X[b,M+N+1,d,r,c])]& [1==X[b,p3,d,r,c] OR 1==X[b,p4,d,r,c] OR 1==X[b,p5,d,r,c]] OR 1==X[b,p6,d,r,c]]=>[Inside_CTV==class(Y[b,0,d,r,c])], (7)

where p3 denotes a channel index for the rectum, p4 denotes a channel index for the mesorectum, p5 denotes a channel index for the internal iliac vessels, and p6 denotes a channel index for the presacral space.

In the second logical constraint, the clause [Middle==axial(X[b, M+N+1, d, r, c])] may be used to determine whether the sample physical point is located in the middle region of the sample subject in the axial direction, which may be equal to 1 if the sample physical point is located in the middle region and 0 if the sample physical point is not located in the middle region. The clause [1==X[b, p3, d, r, c] OR 1==X[b, p4, d, r, c] OR 1==X[b, p5, d, r, c]] OR 1==X[b,p6,d,r,c]] may be used to determine whether the sample physical point belongs to one of the rectum, the mesorectum, the internal iliac vessels, or the presacral space of the sample subject, which may be equal to 1 if the sample physical point belongs to one of the rectum, the mesorectum, the internal iliac vessels or the presacral space, and equal to 0 if the sample physical point doesn't belong to any one of the rectum, the mesorectum, the internal iliac vessels, or the presacral space. The clause [Inside_CTV==class(Y[b, 0, d, r, c])] may be used to determine whether the sample physical point is predicted to be inside the sample CTV the rectum, which may be equal to 1 if the sample physical point is predicted to be inside the sample CTV and 0 if the sample physical point is predicted to be not inside the sample CTV. The implementation of the second logical constraint may be similar to that of the first logical constraint, and the descriptions thereof are not repeated here.

In 904, the processing device 140B (e.g., the construction module 410) may construct a first loss function for evaluating whether the predicted boundary information of a training sample satisfies the one or more logical constraints.

The predicted boundary information of a training sample may be outputted by the preliminary model or an updated preliminary model during model training. The first loss function may be used to evaluate whether the predicted boundary information satisfies the logical constraints. For example, the first contouring guideline for rectal cancer may be converted into the first contouring guideline as defined in Equation (6) as aforementioned. For each sample physical point of a training sample, the processing device 140B may determine the value of the first logical constraint. The first loss function may have a smaller value when the value of the first logical constraint is equal to 1 than when the value of the first logical constraint is equal to 0.

In some embodiments, the one or more logical constraints may include a plurality of logical constraints. The first loss function may incorporate a weight of each of the plurality of logical constraints. For example, a logical constraint having a higher weight may have a greater importance in CTV contouring. Merely by way of example, it is assumed that the contouring guideline is converted into a set of logical constraints R₁, R₂, . . . , and Rₘ, and a weight λi is assigned to an i^{th} logical constraint. For a sample physical point v of a training sample, a sample model input with respect to the sample physical point v is denoted as X(v), a sample output with respect to the sample physical point v is denoted as Y(v), and an evaluation result of whether the sample physical point v satisfies the i^{th} logical constraint is denoted as Rᵢ(X(v),Y(v)) (which may have a value in [0, 1]). The first loss function may be constructed according to Equation (8) as below:

LogicLoss(v)=—(q(v)*log(Y(v)+(1−q(v))*log(1−Y(v)))), (8)

where LogicLoss(v) denotes the value of the first loss function corresponding to the sample physical point v, and q(v) denotes a logic evaluation output relating to the evaluation result of the logical constraints. For example, q(v) may be determined according to Equation (9) as below:

q(v)=Y(v)*e^{(-CΣ}^{ᵢ₌₁}^{^{m}}^{λ}^{ᵢ}^{(1-R}^{ᵢ}^{(X(v),Y(v)))),} (9)

where C represents an adjustment coefficient. In some embodiments, the value of the adjustment coefficient may be adjusted to obtain different loss functions. For example, the value of the adjustment coefficient may be adjusted according to a default setting of the RT system 100, manually by a user, or by processing device 140A according to an actual need.

In 906, the processing device 140B (e.g., the construction module 410) may construct a second loss function for measuring a difference between the predicted boundary information and the ground truth boundary information of a training sample.

Exemplary second loss functions may include a focal loss function, a log loss function, a cross-entropy loss, a Dice ratio, or the like. Merely by way of example, the second loss function may be constructed according to Equation (10) as below:

crossEntropyLOSS(v)=−(GT(v)*(Y(v)+(1−GT(v))*(1−Y(v)), (10)

where crossEntropyLOSS(v) denotes the value of the second loss function corresponding to a sample physical point v, GT(v) denotes the ground truth boundary information relating to the sample physical point v, and Y(v) denotes the predicted boundary information relating to the sample physical point v.

In 908, the processing device 140B (e.g., the construction module 410) may construct the loss function based on the first loss function and the second loss function. For example, the loss function may be a sum or a weighted sum of the first loss function and the second loss function. Merely by way of example, the processing device 140B may construct the loss function according to Equation (11) as below:

Loss(v)=W*LogicLoss(v)+(1−W)*crossEntropyLoss(v), (11)

where Loss(v) denotes the value of the loss function corresponding to a sample physical point v, W denotes the weight of the LogicLoss(v), and (1−W) denotes the weight of the crossEntropyLoss(v).

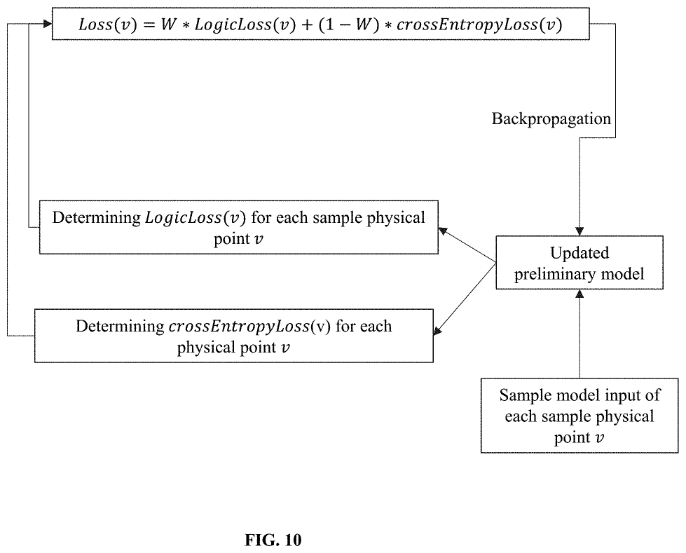

In some embodiments, the loss function may be used in training the preliminary model. For illustration purposes, FIG. 10 illustrates a schematic diagram illustrating an exemplary current iteration for training the preliminary model according to some embodiments of the present disclosure. The current iteration may be performed using one or more target training samples. As shown in FIG. 10, in the current iteration, a sample model input of each sample physical point v of each target training sample may be inputted into an updated preliminary model generated in a previous iteration. The updated preliminary model may output predicted boundary information of each sample physical point v, or other information that needs to be processed by the processing device 140B to generate the predicted boundary information.

The processing device 140B may determine LogicLoss(v) (i.e., the value of the first loss function) and crossEntropyLoss(v) (i.e., the value of the second loss function) for each sample physical point v. The processing device 140B may then determine Loss(v) (i.e., the value of the loss function) for each physical point v based on the LogicLoss(v) and the crossEntropyLoss(v) of the sample physical point v. The processing device 140B may then perform a backpropagation on the updated preliminary model based on the Loss(v) of each physical point v. In some embodiments, the processing device 140B may determine, for example, an average value of the Loss(v) of each sample physical point v as a final value of the loss function, and update the updated preliminary model based on the final value of the loss function.

It should be noted that the above descriptions regarding FIGS. 8-10 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, a logical constraint may be constructed using one or more logical operators different from those defined in Equations (1)-(5). As another example, an Equation provided above may be modified according to an actual need, for example, include one or more additional coefficients or be without one or more of the coefficients as discussed above.

FIG. 11 is a schematic diagram illustrating an exemplary process for determining boundary information relating to a target volume of a subject according to some embodiments of the present disclosure. Process 1100 may be an exemplary embodiment of process 500 as described in connection with FIG. 5.

In 1102, the processing device 140A (e.g., the acquisition module 402) may obtain one or more target images of the subject. The subject may include a target region to which a radiation treatment is directed. Operation 1102 may be performed in a similar manner as operation 502, and the descriptions thereof are not repeated here.

In 1104, the processing device 140A (e.g., the acquisition module 402) may obtain a target volume segmentation model M1 having been trained according to a machine learning technique.

In some embodiments, the target volume segmentation model M1 may be configured to receive the target image(s), and output boundary information relating to the target volume (e.g., a GTV, a CTV, a PTV) as well as boundary information relating to one or more OARs near the target region. Using the target volume segmentation model M1 may improve the efficiency of clinical target contouring and provide more comprehensive information relating to the target region, thereby improving the diagnosis efficiency and accuracy.

In some embodiments, the target volume segmentation model M1 may be generated by training a second preliminary model using a plurality of second training samples. Each of the second training samples may include one or more sample images of a sample subject, ground truth boundary information relating to a sample target volume of the sample subject, and ground truth boundary information relating to one or more sample OARs of the sample subject. The training of the second preliminary model may be performed in a similar manner as that of the preliminary model as described in connection with FIG. 8, and the descriptions thereof are not repeated here.

In 1106, the processing device 140A (e.g., the determination module 404) may determine the boundary information relating to the target volume and the boundary information relating to the OAR(s) based on the target image(s) and the target volume segmentation model M1. Operation 1106 may be performed in a similar manner as operation 506, and the descriptions thereof are not repeated here.

FIG. 12 is a schematic diagram illustrating an exemplary process for determining boundary information relating to a target volume of a subject according to some embodiments of the present disclosure. Process 1200 may be an exemplary embodiment of process 500 as described in connection with FIG. 5.

As illustrated in FIG. 12, in 1202, the processing device 140A (e.g., the acquisition module 402) may obtain one or more target images of the subject. Operation 1202 may be performed in a similar manner as operation 502, and the descriptions thereof are not repeated here.

In 1204, the processing device 140A (e.g., the acquisition module 402) may obtain one or more OAR segmentation models.

An OAR segmentation model may correspond to one or more specific organs or tissues. For example, a brain segmentation model may correspond to the human brain and be used for brain segmentation. Merely by way of example, the brain segmentation model may receive a model input (e.g., target image(s) and/or other information relating to a patient), and output boundary information relating to the brain of the patient. In some embodiments, the OAR segmentation model may include a deep learning model, such as a Deep Neural Network (DNN) model, a Convolutional Neural Network (CNN) model, a Recurrent Neural Network (RNN) model, a Feature Pyramid Network (FPN) model, etc. Exemplary CNN models may include a V-Net model, a U-Net model, a Link-Net model, or the like, or any combination thereof.

An OAR segmentation model may be generated by, for example, the processing device 140B or another computing device (e.g., a processing device of a vendor of the segmentation model) according to a machine learning algorithm as described elsewhere in this disclosure (e.g., FIG. 4B and the relevant descriptions). Merely by way of example, the brain segmentation model may be trained using a plurality of training images of human brain, wherein the brain in each training image has been annotated or confirmed by a doctor. In some embodiments, the obtaining of an OAR segmentation model may be performed in a similar manner as that of the target volume segmentation model as described elsewhere in this disclosure, and the descriptions thereof are not repeated here.

In 1206, the processing device 140A (e.g., the determination module 404) may determine boundary information relating to one or more OARs near the target region based on the target image(s) and the OAR segmentation model(s).

For example, for a specific organ near the target region, the processing device 140A may input the target image(s) into a specific OAR segmentation model corresponding to the specific organ, and the specific OAR segmentation model may output boundary information relating to the specific OAR. In some embodiments, the determination of the boundary information relating to an OAR using an OAR segmentation model may be performed in a similar manner as the determination of the boundary information relating to a target volume using a target volume segmentation model as described in connection with operation 506, and the descriptions thereof are not repeated here.

In 1208, the processing device 140A (e.g., the determination module 404) may determine a stage of the target region.

For example, the processing device 140A may determine the stage of the target region based on the target image(s), the boundary information of the OAR(s) near the target region, and/or other information (e.g., diagnostic information) regarding the target region using a stage determination model. More descriptions regarding the determination of the stage of the target region may be found elsewhere in the present disclosure. See, e.g., operation 604 and relevant descriptions thereof.

In 1210, the processing device 140A (e.g., the acquisition module may obtain a target volume segmentation model M2 having been trained according to a machine learning technique.

In some embodiments, the target volume segmentation model M2 may be a machine learning model that is configured to receive the target image(s), the boundary information relating to the OAR(s), and the stage of the target region, and output boundary information relating to the target volume (e.g., a GTV, a CTV, a PTV). For example, the target volume segmentation model M2 may be generated by training a third preliminary model using a plurality of third training samples. Each of the third training samples may include one or more sample images of a sample subject, a sample stage of a sample target region of the sample subject, sample boundary information relating to one or more sample OARs of the sample subject, and ground truth boundary information relating to a sample target volume of the sample subject. The training of the third preliminary model may be performed in a similar manner as that of the preliminary model as described in connection with FIG. 8, and the descriptions thereof are not repeated here.

In 1212, the processing device 140A (e.g., the determination module 404) may determine the boundary information relating to the target volume based on the target image(s), the boundary information relating to the OAR(s), and the stage of the target region using the target volume segmentation model M2. Operation 1212 may be performed in a similar manner as operation 506, and the descriptions thereof are not repeated here. In some embodiments, the boundary information relating to the OAR(s) and the stage of the target region may provide reference information regarding the target volume and facilitate the segmentation of the target volume.

It should be noted that the above description regarding the processes 1100 and/or 1200 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 500 may be accomplished with one or more additional operations not described and/or without one or more of the operations discussed above.

For example, operation 1208 may be omitted, and the processing device 140A may determine the boundary information relating to the target volume based on the boundary information relating to the OAR(s) and the target image(s) using the target volume segmentation model. Additionally or alternatively, in 1210, the processing device 140A may obtain a target volume segmentation model M2 corresponding to the stage of the target region. As another example, operation 1204 may be omitted, and the boundary information relating to the OAR(s) may be determined manually by a user (doctor).

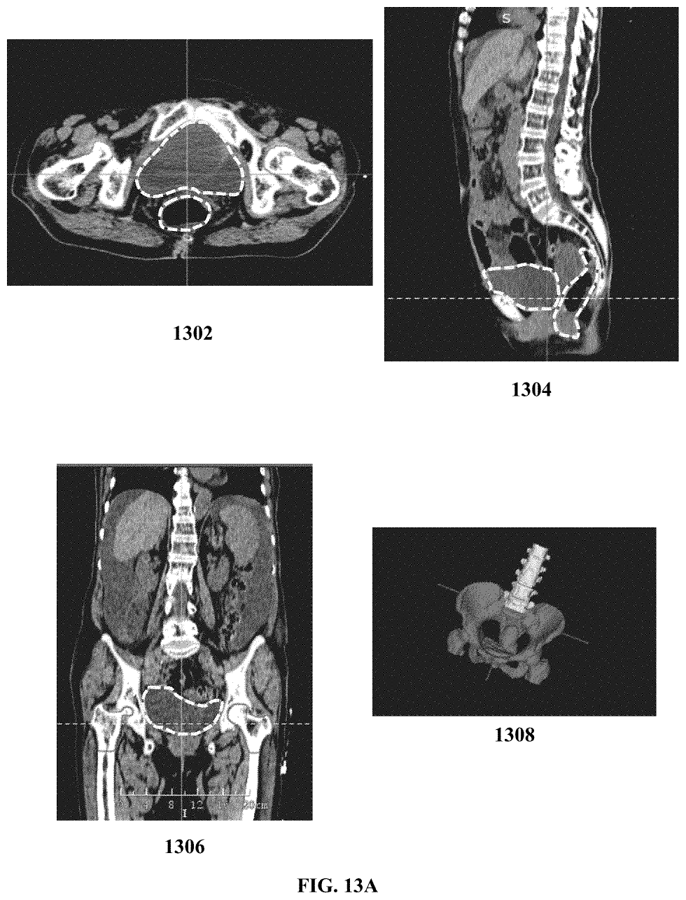

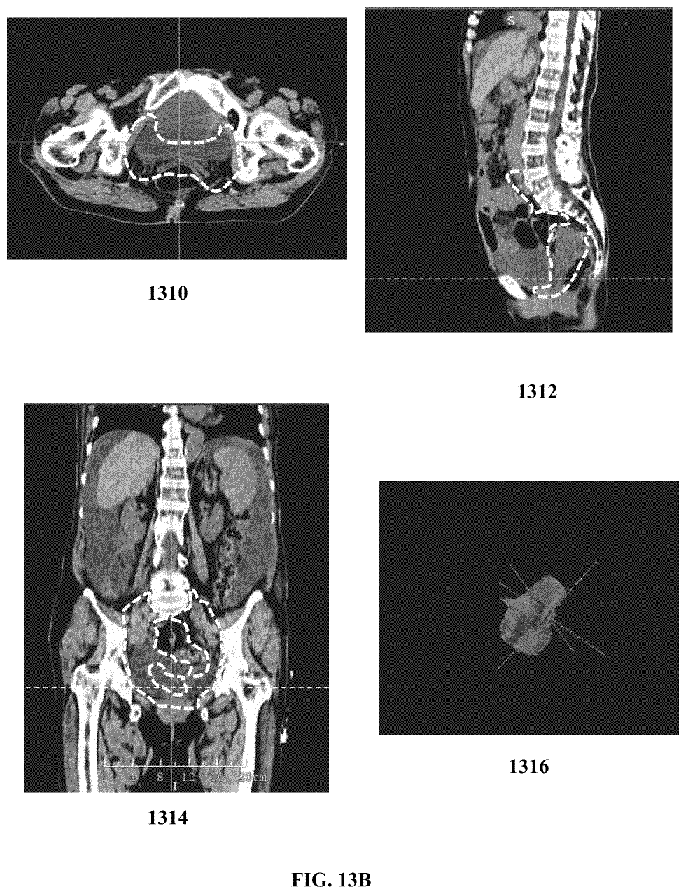

FIG. 13A is schematic diagram illustrating exemplary OAR segmentation images of a subject according to some embodiments of the present disclosure. As shown in FIG. 13A, the OAR segmentation images of the subject include an OAR segmentation image 1302 corresponding an axial plane of the subject, an OAR segmentation image 1304 corresponding to a sagittal plane of the subject, an OAR segmentation image 1306 corresponding a coronal plane of the subject, and a 3D OAR segmentation image 1308 of the subject. In each of the OAR segmentation images 1302, 1304, and 1306, regions representing OARs of the subject (e.g., blood vessels, bones, a bladder, a rectum, and muscles) are annotated by dotted lines.

FIG. 13B is a schematic diagram illustrating exemplary CTV segmentation images of a subject according to some embodiments of the present disclosure. As shown in FIG. 13B, the CTV segmentation images of the subject include a CTV segmentation image 1310 corresponding the axial plane of the subject, a CTV segmentation image 1312 corresponding to the sagittal plane of the subject, a CTV segmentation image 1314 corresponding the coronal plane of the subject, and a 3D CTV segmentation image 1316 of the subject. In each of the CTV segmentation images 1310, 1312, and 1314, regions representing the CTV of the subject are annotated by dotted lines. The CTV segmentation images were generated using a target volume segmentation model disclosed herein.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including a subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

1. A system for clinical target contouring in radiotherapy, comprising:
at least one storage device including a set of instructions; and
at least one processor configured to communicate with the at least one storage device, wherein when executing the set of instructions, the at least one processor is configured to direct the system to perform operations including:
obtaining one or more target images of a subject, the subject including a target region to which a radiation treatment is directed;
obtaining a target volume segmentation model having been trained according to a machine learning technique; and
determining, based on the one or more target images and the target volume segmentation model, boundary information relating to a target volume of the subject, the target volume including at least part of the target region.

2. The system of claim 1, wherein the target volume includes at least one of a gross tumor volume (GTV), a clinical target volume (CTV), or a planning target volume (PTV) of the subject.

3. The system of claim 1, wherein the target volume segmentation model has been trained according to a loss function, the loss function being constructed according to a contouring guideline for delineating the target region and one or more organs at risk (OARs) near the target region.

4. The system of claim 3, wherein
the target volume includes a CTV of the subject,
the target volume segmentation model includes a CTV segmentation model,
the loss function is constructed according to the contouring guideline for delineating the CTV of the target region such that the CTV includes the target region and the OARs near the target region, and
the determining, based on the one or more target images and the target volume segmentation model, boundary information relating to a target volume comprises determining, based on the one or more target images and the CTV segmentation model, boundary information relating to the CTV of the subject.

5. The system of claim 4, wherein the determining, based on the one or more target images and the CTV segmentation model, boundary information relating to the CTV of the subject comprises:
for each of the one or more OARs, obtaining a segmentation image of the OAR;
for each physical point of the subject, obtaining position information of the physical point; and
determining the boundary information relating to the CTV of the subject by processing the one or more segmentation images, the position information, and the one or more target images of the subject using the CTV segmentation model.

6. The system of claim 5, wherein for each physical point of the subject, the position information of the physical point comprises at least one of:
position information of the physical point along a direction perpendicular to an axial plane of the subject,
position information of the physical point along a direction perpendicular to a coronal plane of the subject, or
position information of the physical point along a direction perpendicular to a sagittal plane of the subject.

7. The system of claim 4, wherein the CTV segmentation model is generated by a model training process comprises:
obtaining a preliminary model;
obtaining a plurality of training samples each of which comprises one or more sample images of a sample subject and ground truth boundary information relating to a sample CTV of the sample subject, the sample CTV comprising a sample target region of the sample subject and one or more sample OARs near the sample target region;
constructing, based on the contouring guideline, the loss function; and
generating the CTV segmentation model by training the preliminary model using the plurality of training samples according to the loss function.

8. The system of claim 7, wherein the constructing, based on the contouring guideline, the loss function comprises:
converting the contouring guideline into one or more logical constraints; and
constructing, based on the one or more logical constraints, the loss function.

9. The system of claim 8, wherein the training the preliminary model using the plurality of training samples according to the loss function comprises an iterative operation including one or more iterations, and at least one iteration of the one or more iterations comprises:
for each of at least a portion of the plurality of training samples, obtaining predicted boundary information of the sample CTV of the training sample based on an updated preliminary model generated in a previous iteration;
determining, based on the predicted boundary information of each of the at least a portion of the plurality of training samples, a value of the loss function; and
determining, based on the value of the loss function, an assessment result of the updated preliminary model.

10. The system of claim 9, wherein the constructing, based on the one or more logical constraints, the loss function comprises:
constructing a first loss function for evaluating whether the predicted boundary information of a training sample satisfies the one or more logical constraints;
constructing a second loss function for measuring a difference between the predicted boundary information and the ground truth boundary information of a training sample; and
constructing, based on the first loss function and the second loss function, the loss function.

11. The system of claim 8, wherein the one or more logical constraints comprise a plurality of logical constraints, and the first loss function incorporates a weight of each of the plurality of logical constraints.

12. The system of claim 7, wherein each of the plurality of training samples further comprises:
a sample segmentation image of each of the one or more sample OARs of the sample subject; and
sample position information of each sample physical point of the sample subject.

13. The system of claim 1, wherein the at least one processor is further configured to direct the system to perform the operations including:
generating, based on the boundary information relating to the target volume of the subject, a treatment plan directed to the target region.

14. The system of claim 1, wherein the determining, based on the one or more target images and the target volume segmentation model, boundary information relating to a target volume comprises:
determining, based on the one or more target images, a model input of the target volume segmentation model;
obtaining a model output of the target volume segmentation model by inputting the model input into the target volume segmentation model; and
determining, based on the model output, the boundary information relating to the target volume, wherein the model output includes the boundary information relating to the target volume and boundary information relating to one or more OARs near the target region.

15. The system of claim 1, wherein the determining, based on the one or more target images and the target volume segmentation model, boundary information relating to a target volume comprises:
obtaining boundary information relating to one or more OARs near the target region; and
determining the boundary information relating to the target volume based on the one or more target images, the target volume segmentation model, and the boundary information relating to the one or more OARs.

16. The system of claim 15, wherein the obtaining boundary information relating to one or more OARs near the target region comprises:
obtaining one or more OAR segmentation models; and
determining, based on the one or more target images and the one or more OAR segmentation models, the boundary information relating to the one or more OARs near the target region.

17. The system of claim 15, wherein the determining the boundary information relating to the target volume based on the one or more target images, the target volume segmentation model, and the boundary information relating to the one or more OARs comprises:
determining, based on the one or more target images and the boundary information relating to the one or more OARs, a stage of the target region; and
determining the boundary information relating to the target volume based on the one or more target images, the target volume segmentation model, the boundary information relating to the one or more OARs, and the stage of the target region.

18. A method for clinical target contouring in radiotherapy implemented on a computing device having at least one processor and at least one storage device, the method comprising:
obtaining one or more target images of a subject, the subject including a target region to which a radiation treatment is directed;
obtaining a target volume segmentation model having been trained according to a machine learning technique; and
determining, based on the one or more target images and the target volume segmentation model, boundary information relating to a target volume, the target volume including at least part of the target region.

19. The method of claim 18, wherein the target volume includes at least one of a gross tumor volume (GTV), a clinical target volume (CTV), or a planning target volume (PTV) of the target region.

20-21. (canceled)

22. A non-transitory computer readable medium, comprising a set of instructions for clinical target contouring in radiotherapy, wherein when executed by at least one processor, the set of instructions direct the at least one processor to effectuate a method, the method comprising:
obtaining one or more target images of a subject, the subject including a target region to which a radiation treatment is directed;
obtaining a target volume segmentation model having been trained according to a machine learning technique; and
determining, based on the one or more target images and the target volume segmentation model, boundary information relating to a target volume, the target volume including at least part of the target region.
